(19) United States
(12) Patent Application Publication
(10) Pub. No.: US 2005/0124013 A1
(43) Pub. Date: Jun. 9, 2005

(54) On-line apparatus and method for determining endotoxin levels

(75) Inventors: Matthew R. Bonen, New Market, MD (US); Ronald Norman Berzofsky, Timonium, MD (US); Bruce J. Richardson, Los Gatos, CA (US); Robert L. Lathrop III, Santa Clara, CA (US); Bruce Rodney den Dulk, Ripon, CA (US)

Correspondence Address: BANNER & WITCOFF, 1001 G STREET N W, SUITE 1100, WASHINGTON, DC, 20001, US

(73) Assignee: Cambrex Bio Science Walkersville, Inc., Walkersville, MD (US)

(21) Appl. No.: 10/983,087

(22) Filed: Nov. 8, 2004

Related U.S. Application Data
(60) Provisional application No. 60/518,003, filed on Nov. 7, 2003.

(51) Int. Cl. G01N 33/554; G01N 33/569; C12M 1/34

(52) U.S. Cl. 435/7.32; 435/287.1

(57) ABSTRACT

An apparatus and method for on-line testing for the presence of an endotoxin within a fluid sample from a fluid line. The apparatus is positioned in fluid communication with a fluid line to perform the on-line fluid testing for the presence of at least one endotoxin. The apparatus can include a housing and a fluid sampling system positioned in fluid communication with the fluid line. The fluid sampling system can comprise a valve for controlling the fluid flow from the fluid line into the fluid sampling system. A fluid flow well is positioned within the housing and in fluid communication with the fluid sampling system. A removable assembly can also be secured within the housing. The removable assembly comprises a plurality of wells for receiving used and unused fluid carrying members that can receive samples from the fluid flow well, a plurality of fluid sample receiving wells, and a plurality of vessel retention positions comprising recesses for securely receiving portions of respective fluid vessels. A detecting system is provided for testing a control sample and a sample of the fluid from the fluid line. The results of these tests are compared in order to determine if the fluid sample is carrying any endotoxins. In an embodiment, fluorescence testing of the sample is compared to that of the control in order to determine if the sample includes an endotoxin.

### RELATED APPLICATION

This application claims benefit under 37 CFR §1.78 of provisional application 60/518,003, filed Nov. 7, 2003. The full disclosure of the application is incorporated herein by reference.

### FIELD OF THE INVENTION

An apparatus and method for determining trace endotoxin levels within a fluid, more particularly, an apparatus for positioning in fluid communication with a fluid line and method for on-line determinations of endotoxin levels in fluids.

### BACKGROUND OF THE INVENTION

Bacterial endotoxin is a potentially widespread contaminant of a variety of materials, such as water, food, pharmaceutical products, and parenteral preparations. Bacterial endotoxins (lipopolysaccharides) are released from the outer cell membranes of Gram-negative bacteria during early stages of growth, phagocytic digestion, or autolysis of bacterial cells. Lipopolysaccharides are water-soluble stable molecules that have both hydrophobic and hydrophilic regions. The latter are composed of repeating oligosaccharide side chains attached to a polysaccharide core.

There is considerable variation in the details of the structure of endotoxins derived from different bacteria. While the polysaccharide moiety is responsible for the immunogenic properties of endotoxins, their toxicity is elicited by the hydrophobic part (called ‘lipid A,’ which is virtually invariant in composition across different bacterial species). Even in small doses, the introduction of endotoxins into the circulatory system of either humans or animals is capable of causing a wide spectrum of nonspecific pathophysiological changes, e.g., fever, increased erythrocyte counts, disseminated intravascular coagulation, hypotension, shock, cell death, etc. In large doses, it causes death in most mammals. Early-life exposure to endotoxins exerts long-term effects on endocrine and central nervous system development and increases predisposition to inflammatory diseases. Shanks et al., Proc. Natl. Acad. Sci. 97, 5645-50, 2000; see also Pearson III, in PYROGENS: ENDOTOXINS, LAL TESTING, AND DEPYROGENATION, Pearson III, ed., Marcel Dekker, Inc., NY, 1985, pp. 11-19; URL address http file type, www host server, domain name “bact.wisc.edu,” file name “Bact330/lectureendo/.”

Given current concerns regarding bioterrorism, it is useful to note that inhalation of high concentration of endotoxins causes dry cough and shortness of breath, accompanied by a decrease in lung function and fever. Rylander, in ORGANIC DUSTS: EXPOSURE, EFFECTS AND PREVENTION, Rylander & Jaccobs, eds., Lewis Publishers, Boca Raton, Fla., 1994; Heederik & Douwes, Ann. Agric. Environ. Med. 4, 17-19, 1997. Epidemiological and animal studies show that chronic respiratory exposure to endotoxins may lead to chronic bronchitis and reduced lung function. Rylander, Scand. J Work Environ. Health 11, 199-206, 1985.

It is thus essential to ensure that the endotoxin contents of parenterally administered drugs or other fluids remain below permissible levels (in the US, this is set by the US Food and Drug Administration). Sterile water for injection or irrigation, for example, has a maximum permissible limit of 0.25 Endotoxin Units (EU)/mL (for endotoxin derived from E. coli, 1 EU is approximately 75-200 pg). See the URL address: http file type, www host server, domain name “fda.gov,” file type “ora/inspect_ref/itg/itg40.html”; United States Pharmacopeia, USP 24-NF 19, Suppl. 2, 2761-62; Jul. 1, 2000.

Measurement of Endotoxins

The rabbit pyrogen test (fever induction in a rabbit) was introduced in the U.S. Pharmacopoeia in 1942 for the general testing of pyrogens, which include bacterial endotoxins. The test is slow and qualitative and has largely been replaced by some form of the Limulus amebocyte lysate (LAL) test. In 1964, Levin and Bang discovered that bacterial endotoxins can greatly accelerate the rate of clotting of blood from the horseshoe crab Limulus polyphemus. Levin & Bang, Bull. Johns Hopkins Hosp. 115, 265-74, 1964; see also the URL address: http file type, www host server, domain name “dnr.state.md.us,” file type “fisheries/education/horseshoe/horseshoefacts.html.” By 1987, the US Food and Drug Administration (FDA) published guidelines for the validation of the LAL test as an alternative to the USP Rabbit Pyrogen Test. The superiority of the LAL based assay over the rabbit test has been known for some time. See Levin, in ENDOTOXINS AND THEIR DETECTION WITH THE LIMULUS AMEBOCYTE LYSATE TEST, Watson et al., eds., Alan R. Liss, Inc., NY, 1982, 7-24. Berzofsky U.S. Pat. No. 5,310,657 clearly showed that the LAL test is two orders of magnitude more sensitive than the rabbit test and also less expensive, less time consuming, and easier to perform.

LAL contains several protease enzymes responsible for endotoxin induced gel/clot formation. Through a series of cascade reactions, the primary protein component sensitive to endotoxins activates the proclotting enzyme to form the clotting enzyme. Berzofsky & McCullough in IMMUNOLOGY OF INSECTS AND OTHER ARTHROPODS, Gupta, ed., CRC Press, Boca Raton, Fla., 1991, pp. 429-48; Morita et al., Haemostasis 7, 53-64, 1978. The clotting enzyme then transforms coagulogen to coagulin, which self-associates to form a gel.

Presently there are three major versions of LAL tests: the gel-clot assay (Levin & Bang, 1964; Levin, 1982; U.S. Pat. No. 5,310,657), the turbidimetric assay (Levin et al., J. Lab. Clin. Med. 75, 903-11, 1970; Cooper et al., J. Lab. Clin. Med. 78, 138-48, 1971; Pearson & Weary, J. Lab. Clin. Med. 78, 65-77, 1971); and the colorimetric assay (Teller & Kelly, in BIOMEDICAL APPLICATION OF THE HORSE SHOE CRAB (LIMULIDAE), Cohen, ed., Alan R. Liss Inc., NY, 1979, 423-34; Ditter et al., J. Lab. Clin. Med. 78, 65-77, 385-92, 1971; Dubczak et al., Haemostasis 7, 403-14, 1978; Novitsky & Roslansky, in BACTERIAL ENDOTOXINS: STRUCTURE, BIOMEDICAL SIGNIFICANCE, AND DETECTION WITH THE LIMULUS AMEBOCYTE LYSATE TEST, Cate et al., eds., Alan R. Liss, Inc., NY, 1985, 181-93; Sturk et al., Haemostasis 7, 117-36, 1978; Iwanaga et al., Haemostasis 7, 183-88, 1978; Tsuji & Martin, Haemostasis 7, 151-66, 1978; Tsuji et al., Appl. Env. Microbiol. 48, 550-55, 1984).

Turbidimetric assays measure turbidity due to gel formation; apparent turbidity is somewhat affected by the size and the number of particles, etc. but this problem can be largely overcome. Ohki et al., FEBS Lett. 120, 217-20, 1980. Turbidity measurement is generally unaffected by color present in the sample. A quartz oscillator has been used to measure the viscosity change that occurs during gelation; this technique allows turbid samples to be analyzed. Novitsky et al., in DETECTION OF BACTERIAL ENDOTOXINS WITH THE LIMULUS AMEBOCYTE LYSATE TEST, Watson et al., eds., Alan R. Liss, Inc., NY, 1987, pp 189-96.

In a colorimetric assay, a synthetic chromogenic peptide is hydrolyzed by the clotting enzyme to release the terminal colored chromogenic moiety. It provides better quantitation and is less laborious than clotting based methods. It is also more sensitive because the amount of enzyme needed for the hydrolysis of the chromogenic substrate is less than the amount needed to form a clot. Friberger et al., in ENDOTOXINS AND THEIR DETECTION WITH THE LIMULUS AMEBOCYTE LYSATE TEST, pp 195-206.

Turbidimetric and colorimetric assays can be practiced in two modes. In the endpoint mode, turbidity or color is measured after a fixed incubation period. In the kinetic assay mode, which offers greater dynamic range, the turbidity or color development is measured continuously as a function of time. In the end point assay mode, a calorimetric reaction can be stopped by adding acid or a surfactant solution (e.g., SDS), and the absorbance can be measured at any time thereafter. In a turbidimetric assay this is not possible; addition of acid also destroys the turbidity.

Automation

A degree of automation of the turbidimetric end point assay has been achieved with a commercially available system (Muramatsu et al., Anal. Chim. Acta 215, 91-98, 1988; Homma et al., Anal. Biochem. 204, 398-404, 1992); however, poor correlation with other methods and generally higher results have been observed (Tsuji & Martin, 1978).

For some time now, the chromogenic LAL test is the most widely used. Jorgensen & Alexander, Appl. Environ. Microbiol. 41, 1316-20, 1981; Novitsky et al., Parenteral. Sci. Technol. 36, 11-16, 1982.

A robotic automated system has been developed for the chromogenic test. Tsuji & Martin, 1978. This early system and its subsequent commercial counterparts has impressive capabilities but the overall cost is very high. See Bussey & Tsuji, J. Parenter. Sci. Technol. 38, 228-33, 1984; Martin et al., J. Parenter. Sci. Technol. 40, 61-66, 1986. In fact, the cost is prohibitive for deployment at each point of use, as is necessary, for example, in sterile water testing applications. Rather, most users utilize microplate reader based instrumentation where 96-well plates are manually loaded with samples, standards, and reagents. See the URL address: http file type, www host server, domain name “Cambrex.com,” file name “biosciences/lal/b-EndotoxinDPS-instrument.htm# 1.”

It is known in the art to use flow injection analysis or sequential injection analysis when attempting to detect the presence of a species. Conventional sequential injection analysis involves the use of a system comprising, typically, a rotary, multi-position selection valve around which multiple liquid solutions including samples and reagents are arranged. A bi-directional pump is used to draw up volumes of these samples and reagents through respective ports of the selection valve and into a holding coil where the samples and reagents are stacked and then delivered to a detector for analysis. This process causes mixing of the sample and reagent segments leading to chemistry that forms a detectable species before reaching the detector. The detector is typically attached to one port of the rotary valve via which the stacked segments can be made to flow by the pump. Stacking is the process of providing a plurality of aliquots, slugs or segments of fluids in a single conduit, either discrete and apart one slug or aliquot from another or adjacent to one another. Conventional systems can involve the use of a single pump (syringe or peristaltic) and a single rotary selection valve. Conventional multi-position selection valves permit random access of the ports that are connected to the samples, the reagents and the detector. Conventional selection valves that are usable in sequential injection analysis systems are can have between six and twenty-eight ports. Commonly, the section valves have between eight and ten ports. An electronic actuator that, in some instances, moves through the ports in both clockwise and counter-clockwise directions controls the operation of the selection valve. Typically, only one port is accessed at any time. When compared to flow injection analysis, sequential injection analysis systems have the advantage of being able to access an increased number of solutions with just one pump. However, these types of sequential injection analysis systems have not been used to determine the presence of the endotoxins due, at least in part, to the difficulties in cleaning the system between different test samples.

There is, therefore, a need in the art for an affordable, sensitive, and fully automated (“on-line”) endotoxin determination system that can be used for point of use endotoxin determinations with a fluid line.

### SUMMARY OF THE INVENTION

Aspects of the present invention include an apparatus and method for on-line testing for the presence of an endotoxin within a fluid sample from a fluid line. The sampling and analysis can occur while the fluid line is in operation. Also, the testing can occur by diverting part of the fluid in the line without having to shutdown or interrupt the operation of the fluid line.

The apparatus is positioned in fluid communication with a fluid line to perform the on-line fluid testing for the presence of at least one endotoxin. The apparatus can include a housing and a fluid sampling system positioned in fluid communication with the fluid line. The fluid sampling system can comprise a valve for controlling the fluid flow from the fluid line into the fluid sampling system. A fluid flow well is positioned within the housing and in fluid communication with the fluid sampling system. A removable assembly can also be secured within the housing. The removable assembly comprises a plurality of wells for receiving used and unused fluid carrying members that can receive samples from the fluid flow well, a plurality of fluid sample receiving wells, and a plurality of vessel retention positions comprising recesses for securely receiving portions of respective fluid vessels. A detecting system is provided for testing a control sample and a sample of the fluid from the fluid line. The results of these tests are compared in order to determine if the fluid sample is carrying any endotoxins. In an embodiment, fluorescence testing of the sample is compared to that of the control in order to determine if the sample includes an endotoxin.

The method for performing on-line detection of an endotoxin within the fluid carried by the fluid line can include the steps of positioning an endotoxin testing apparatus within the fluid line of the fluid system and directing fluid from the fluid line into the testing apparatus. The method can also include sampling the directed fluid and delivering the sample to a receiving well. Additionally, the method can include the steps of obtaining an endotoxin identifying agent, introducing the agent into the receiving well containing the fluid sample and detecting the presence of any endotoxin within the sample.

### BRIEF DESCRIPTION OF THE FIGURES

FIG. 1 is a schematic drawing of a fluid line system including an on-line endotoxin detecting apparatus according to aspects of the present invention;

FIG. 2 is a perspective view of the endotoxin detecting apparatus illustrated in FIG. 1;

FIG. 3 illustrates a fluid sampling system that forms a portion of the apparatus illustrated in FIG. 2;

FIG. 4 illustrates an alternative embodiment of the fluid sampling system illustrated in FIG. 2;

FIG. 5 is an exploded view of a fluid sampling system illustrated in FIG. 4;

FIG. 6 illustrates a removable assembly shown in FIG. 2;

FIG. 7 is a partial illustration of a detecting system that forms a portion of the apparatus illustrated in FIG. 2;

FIG. 8 illustrates a sensor arrangement of the detecting system of FIG. 7;

FIG. 9 is a partially broken view of a portion of the assembly of FIG. 6;

FIG. 10 is an exploded view of a cartridge illustrated in FIG. 6;

FIG. 11 illustrates a portion of the cartridge for securely retaining fluid vessels;

FIG. 12 is a partially broken view of a cap retention portion and a vial retention portion of the cartridge;

FIG. 13 is a cross section of a vessel retaining region of the cartridge illustrated in FIG. 11;

FIG. 14 illustrates the cartridge including a cover;

FIGS. 15 and 16 illustrate portions of the undersurface of the cartridge;

FIG. 17 illustrates the cartridge assembly, positioning system and detecting system illustrated in FIG. 2;

FIG. 18 is a partial isometric view of the positioning system;

FIGS. 19A and 19B are isometric views of portions of the positioning system;

FIG. 20 is an isometric view of a portion of the positioning system;

FIGS. 21-23 illustrates steps for removing a cap form a fluid vessel according to an aspect of the present invention;

FIGS. 24 and 25 are isometric views of a system for obtaining and ejecting fluid carrying members;

FIG. 26 is an isometric view of the detecting system of FIG. 2; and

FIGS. 27-29 are isometric views of the heating and cooling portions of the cartridge assembly illustrated in FIG. 2.

### DETAILED DESCRIPTION

The invention provides automated endotoxin detection systems (i.e., automated “on-line” flow analysis systems) that can perform a Limulus amebocyte lysate (LAL)-chromogenic substrate kinetic assay for the determination of bacterial endotoxins. The systems can be used to test fluid samples from production lines to detect the presence of endotoxin during the preparation of, for example, water, food, drink, pharmaceutical products (including those for animal and human health), and parenteral preparations.

In systems of the invention, a test fluid sample is mixed with agent(s), such as a chromogenic substrate and an LAL reagent in a well to form an assay mixture at the point of use. Assay mixtures are then tested to detect the presence of an endotoxin and its level of concentration. An automated system of the invention determines endotoxin concentration with good accuracy and reproducibility in the range of 0.01 to 10 endotoxin units (EU)/mL (r²≧0.99). The automated systems of the present invention have performed using a standard curve from 0.05 EU/mL to 5 EU/mL. A manual system according to the present invention determines endotoxin concentrations with good accuracy and reproducibility in the range of 0.005 to 50 endotoxin units (EU)/mL (r²≧0.99). Based on three times the standard deviation of a blank and the slope of a calibration curve, systems of the invention can detect endotoxin concentrations of 0.003 EU/mL or lower. The variability of the assay method is less than 20% (n=10). Analysis time required for a 0.05 EU/mL standard typically is less than 100 minutes. For example, the analysis time can be about 60 minutes.

LAL Reagent and Chromogenic Substrate

“LAL reagent” as used herein refers both to amebocyte lysates obtained from horseshoe crabs (e.g., Limulus polyphemus, Carcinoscorpius rotundicauda, Tachypleudus tridentata, or Tachypleudus gigas) and to “synthetic” LAL reagents. Synthetic LAL reagents include, for example, purified horseshoe crab Factor C protein (naturally occurring or recombinant) and, optionally, a surfactant, as described in WO 03/002976. One such reagent, “PyroGene™,” is available from Cambrex Bio Science Walkersville, Inc. Reagents such as that discussed in U.S. patent application Ser. No. 10/183,992, published as U.S. Patent Publication No. 20030054432, can be used herein. LAL reagents preferably are obtained from Cambrex Bio Science Walkersville, Inc. Lyophilized LAL reagent can be reconstituted with 1.4 mL of LAL reagent water (endotoxin-free water) and kept refrigerated until use.

Any chromogenic substrate that can be used to detect an active serine protease (thrombin, trypsin, etc.) (i.e., has the sequence “Arg-chromogenic substrate) can be used in the automated systems disclosed herein. Such substrates are well-known and are commercially available. For example, the buffered chromogenic substrate (p-nitroaniline terminated pentapeptide (Ac-Ile-Glu-Ala-Arg-pNA, S50-640) is suitable and can be reconstituted with LAL reagent water and stored under refrigeration until use. Fluorogenic substrates having the sequence “Arg-fluorogenic substrate” also can be used and are encompassed within the term “chromogenic substrate.”

E. coli 055:B5 lyophilized endotoxin obtained from Cambrex Bio Science Walkersville, Inc. can be used to generate standard curves. Typically, lyophilized endotoxin is reconstituted with endotoxin-free water (LAL reagent water, Cambrex Bio Science Walkersville, Inc.) and vortexed for at least five minutes to yield a concentration of 50 EU/mL. Refrigerated reconstituted endotoxin is stable for at least one month. For the preparation of working standards, the stock solution is warmed to room temperature, vortexed for 5 minutes, diluted with LAL reagent water, and vortexed again before use.

Lysate-substrate reagents for use in chromogenic assays typically consist of a mixture of amebocyte lysate and substrate, which is supplied as a co-lyophilized solid in sterile containers. Immediately before use, the user or a robotic system reconstitutes the lysate-reagent by adding a prescribed amount of endotoxin-free reagent water. Equal amounts of the reconstituted reagent and a test sample are pipetted into microplate wells using standard sterile techniques, and the absorbance is monitored as a function of time. A plot of the logarithm of the time t for the starting absorbance to increase by a fixed amount (typically 0.2 AU) vs. log [endotoxin] is linear with a negative slope (color develops faster as the endotoxin concentration increases). The endotoxin concentration of a sample is determined by reference to a calibration curve generated with endotoxin standards and the same reagent batch, usually on the same microplate.

In systems such as those disclosed herein, the LAL reagent and chromogenic substrate should be reasonably stable. Preferably, these components are kept in separate vessels until their combination at the point of use increases stability of these components.

### pH and Temperature for the Chromogenic LAL Assay

According to the literature, the optimum pH for the activation of the LAL reagent is 7.5, while that for the enzymatic cleavage of pNA from the substrate is 8.2-8.5 (Tsuji et al., Appl. Env. Microbiol. 48, 550-55, 1984; Bussey & Tsuji, J. Parenter. Sci. Technol. 38, 228-33, 1984; Dunér, J. Biochem. Biophy. Meth., 26, 131-42, 1993). In a single mixed solution, the optimum pH is 7.7-7.8; the sensitivity is constant in this region (Dunér, 1993). The optimum temperature for the chromogenic LAL assay has been investigated by several researchers and reported to be 37° C. (Bussey & Tsuji 1984; Dunér, 1993). We found that these reported optima apply to the systems disclosed herein as well.

### DESCRIPTION OF THE FIGURES

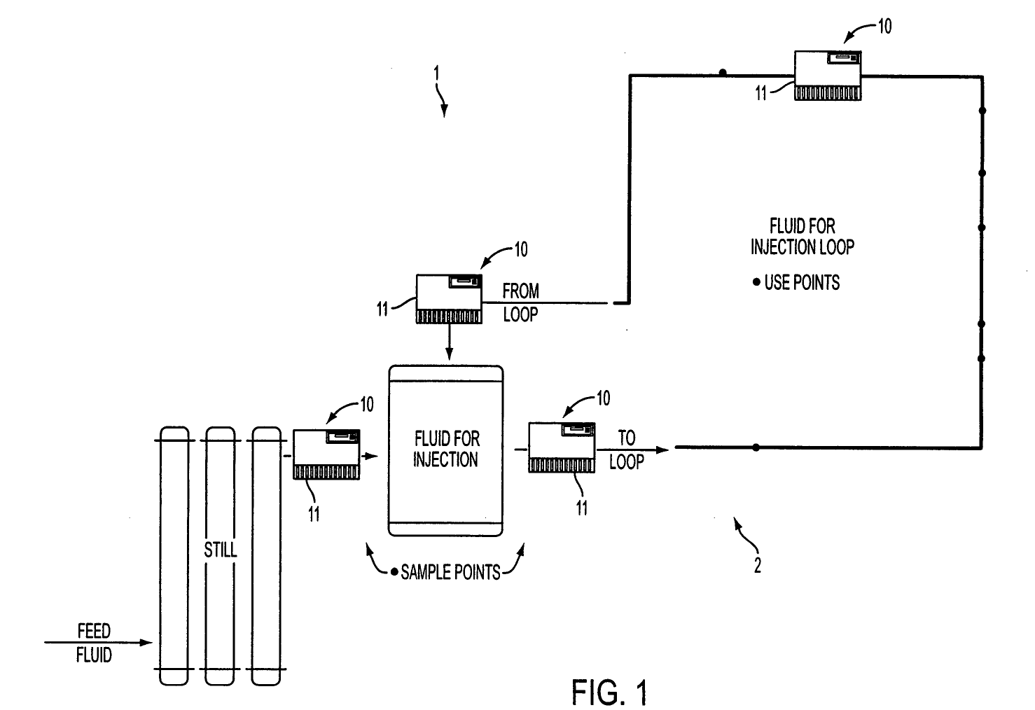

Aspects of the present invention relate to a method and an automated apparatus 10 for performing on-line testing of a fluid to determine endotoxin concentrations. In an embodiment shown in FIG. 1, the fluid tested is water within a process loop 2 such as a WFI or high purity water system 1. In an embodiment, the automated apparatus 10 monitors endotoxins within the water using agents, such as LAL or a recombinant endotoxin moiety, through the use of a chromogenic or fluorogenic detection scheme.

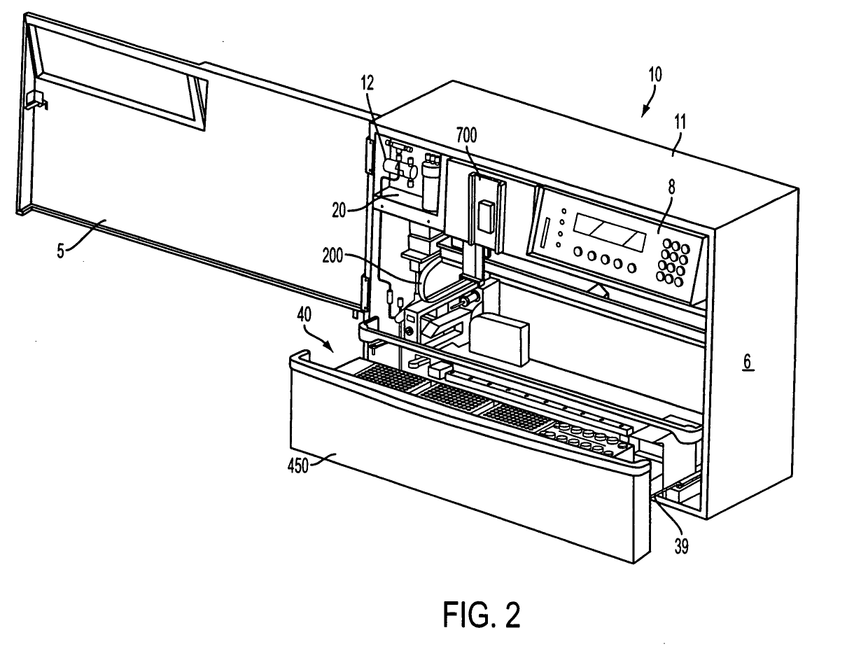

As shown in FIG. 2, the apparatus for determining endotoxin concentrations 10 comprises a housing 11 with an opening 12 for receiving water within the fluid line of the fluid process loop 2 of the water system 1. The housing 11 can include a door 5 that is latched to a frame portion 6 of the housing 11. The door 5 and frame portion 6 can include an optic or contact sensor to determine if the door 5 is properly closed and locked. Also, electronic controls 8 for the apparatus 10 can be positioned on the housing 11 and spaced from the door 5 so that the electronic controls 8 are easily accessed when the door 5 is open.

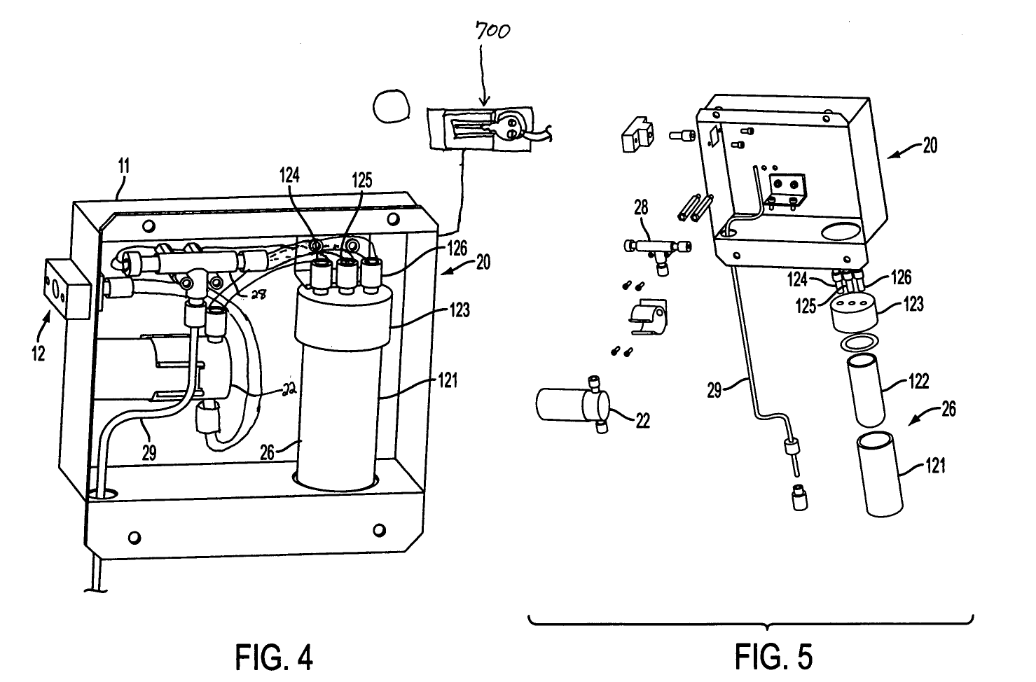

As shown in FIG. 4, a flow path 16 extends between the opening 12 and a fluid sampling delivery system 20. The flow path 16 can include a rigid or flexible fluid delivery tube 17 or other type of conventional fluid delivery conduit, such as a pipe. In an embodiment, the flow rate with the flow path 16 is adjustable between about 0 ml/min to about 100 ml/min.

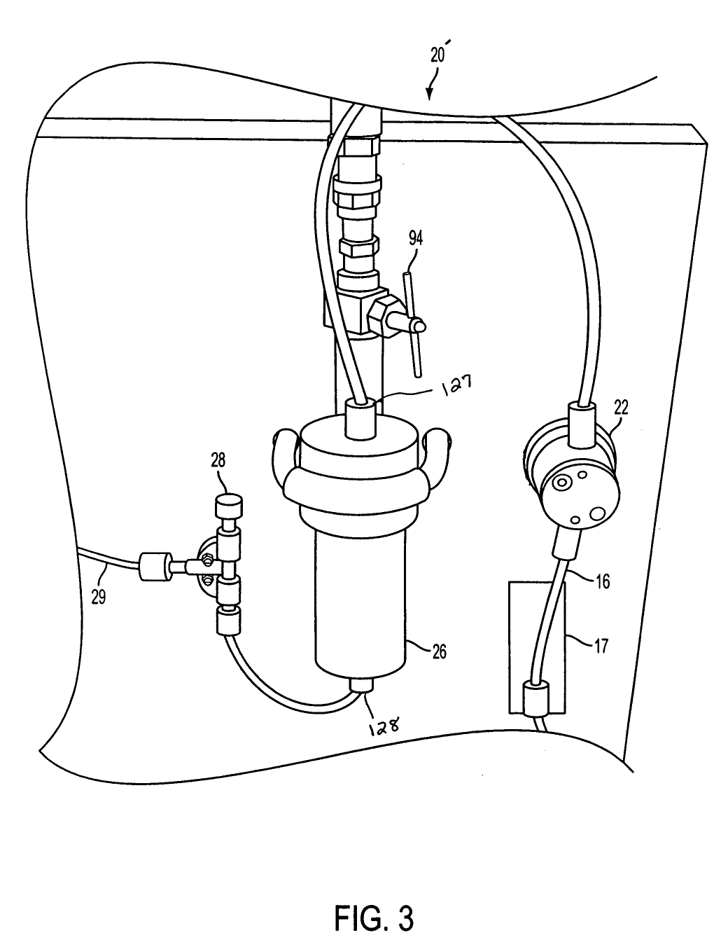

The fluid sampling delivery systems 20′ and 20, shown in FIGS. 3-5, each includes a solenoid valve 22 that opens and closes to control the flow of water into a fluid storage tank 26. The solenoid valve 22 has a preset lower limit at which it opens and a preset upper limit at which it closes. The upper limit of the solenoid valve 22 can be set between about 10 psi and 80 psi. In another embodiment, the upper limit can be set between about 15 psi and 55 psi. In a further embodiment, the upper limit at which the solenoid valve 22 opens can be set at about 16 psi. The lower limit of the solenoid valve 22 can be set between about 1 psi and 20 psi. In another embodiment, the solenoid valve 22 can have a lower limit between about 5 and 15 psi. In yet another embodiment, the lower limit at which the solenoid valve 22 closes is set at about 6 psi. The system is relatively insensitive to the fluid pressure within the water loop 2. Sampling system 20 can be used with water loops having pressures up to, or in excess of, eighty psi.

The fluid storage tank 26 that will contain fluid entering sampling system 20 is positioned downstream from the solenoid valve 22, as shown in FIG. 4. The fluid storage tank 26 can have Teflon or other types of lining materials that prevent the endotoxins from binding to the inner surface of the tank 26. As shown in FIG. 5, the tank 26 includes an outer tank 121, an inner tank 122 and a cover 123 that is positioned over the top of the inner and outer tanks 121 and 122. The cover 123 includes a plurality of input and output ports. As shown in FIG. 4, the cover 123 can include three input/output ports 124, 125 and 126. In other embodiments, the cover 125 can include less than three ports or more than three ports. In another embodiment, illustrated in FIG. 3, the tank 26 includes an input opening 127 at its upper end and an output opening 128 at its lower, downstream end.

FIG. 4 illustrates a metering fluid control valve 28 is located downstream from the tank 26. In one embodiment, the fluid flow control valve 28 is a pinch valve. The fluid control valve 28 is set to control the flow out of the tank 26 so that a substantially continuous flow exits the tank 26 and flows into a fluid delivery conduit 29 for delivering the fluid sample to a fluid flow well 30. The fluid conduit 29 can include a pipe, tube or other known fluid carrying conduit. The fluid flow control valve 28 is set at a pressure that is lower than the lower limit of the solenoid valve 22 so that the pressure within the tank 26 is always greater than pressure maintained by valve 28. As a result, fluid will substantially continuously flow from the tank 26 and into the well 30. In an embodiment, the terminal, downstream end of the fluid conduit 29 is spaced above the opening of the well 30 so that the liquid, such as water, exiting the downstream end of the fluid conduit 29 drops into the well 30. A gauge shutoff valve 94 can be positioned in the flow path at any point between the opening 12 and the well 30.

In operation, the fluid sample received from the fluid line of loop 2 will move into the flow path 16 (FIG. 4). The solenoid valve 22 will remain closed until the lower limit pressure of the solenoid valve 22 is reached within the tank 26. This pressure will be lower than the pressure within the water line 2 of the system 1 being tested. When the lower pressure limit of the solenoid valve 22 is reached, the solenoid valve 22 opens and fluid from within the flow path 16 moves into the tank 26. Then when pressure in the tank 26 reaches the upper limit of the solenoid valve 22, the solenoid valve 22 closes until the pressure within the tank 26 reaches the lower limit as a result of fluid passing out of the downstream end of the tank 26 and past the fluid control valve 28 into the well 30. As will be understood, the pressure within the fluid delivery conduit 29 created by the fluid control valve 28 is less than the lower limit of the solenoid valve 22 so that continuous flow occurs through the fluid delivery conduit 29 when the tank 26 is draining and being filled.

The portions of the embodiments of the above-discussed fluid sample delivery system 20 that contact the water to be tested can be covered or lined along at least their inner surfaces with a Teflon or PE material in order to prevent the binding of the endotoxins from attaching to the wetted surfaces of the parts of the flow path within the system 20.

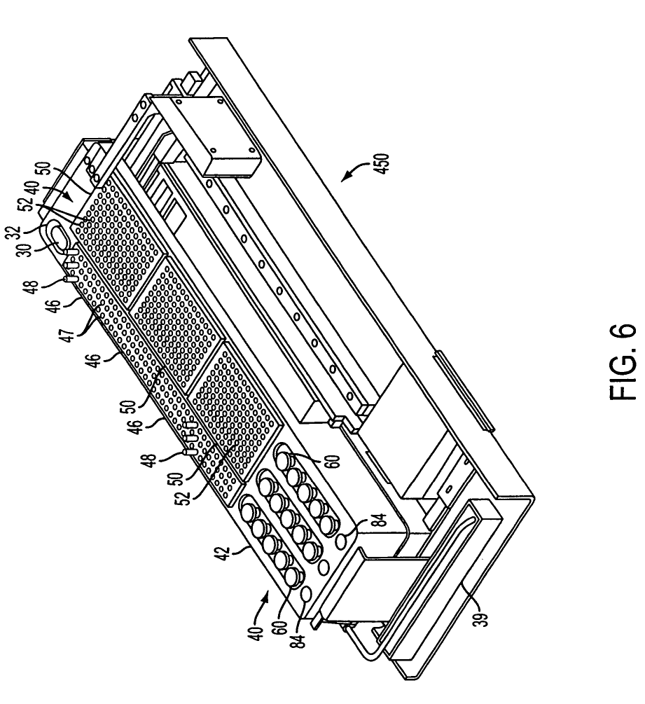

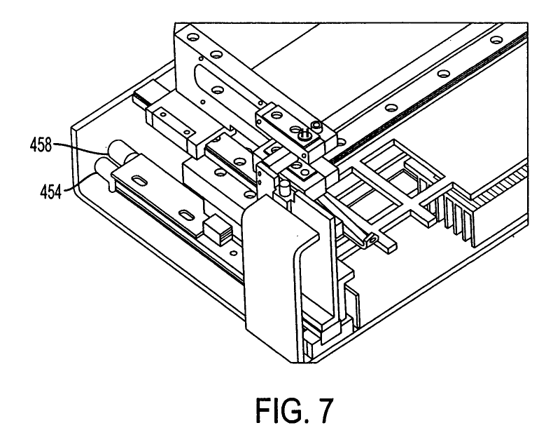

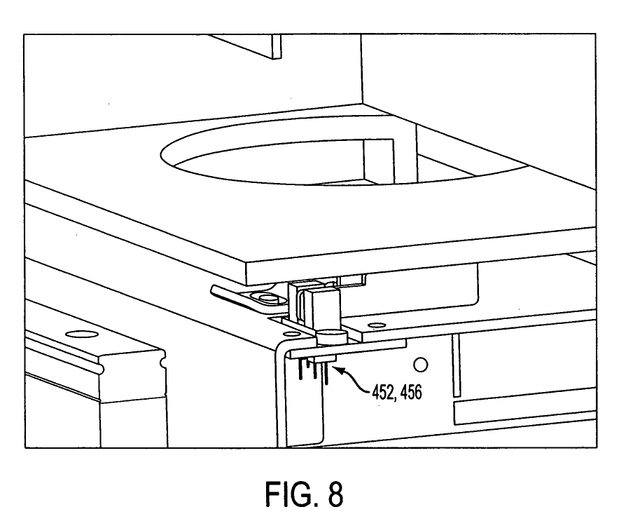

As shown in FIG. 6, the fluid flow well 30 is positioned within a replaceable cartridge assembly 40 of the apparatus 10. The cartridge assembly 40 is removably and replaceably positioned within a moveable drawer 450 (FIG. 2) so that new cartridge assemblies 40 can be positioned within the drawer when a carried cartridge assembly is spent. The drawer 450 is slidably positioned within the housing 11 as illustrated in FIG. 2. The housing 11 can also include an optic or contact sensor 452 (FIG. 8) to determine if the removable drawer 450 is closed. The housing 11 also includes a magnetic detent 454 that permits the accurate and repeatable positioning of the drawer 450 during closure (FIG. 7). A solenoid lock 456 (FIG. 8) can be included for preventing the drawer 450 from unintentionally opening during the operation of the assembly 10. In the embodiment illustrated in FIG. 7, the housing 11 includes at least one dampening member 458 that dampens the movement of the drawer 450 as it moves into and assumes the proper closed position when, for example, the cartridge assembly 40 has been replaced.

The cartridge assembly 40 includes a cartridge housing 42 that has a plurality of openings for removably receiving a plurality of members that can be used during the testing procedures including packaging reagents, pipette tips, microplates and a disposable water sampling well. In an embodiment, the cartridge assembly 40 can be formed of a disposable plastic package.

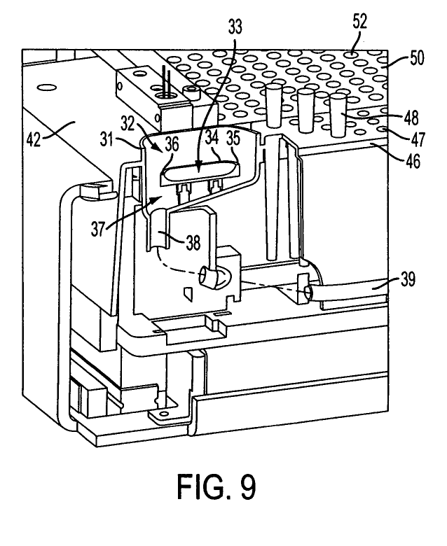

As shown in FIG. 9, the cartridge housing 42 has a first opening 32 that defines an outer fluid well opening of the well 30 through which the fluid being tested enters the well 30. The well 30 also includes an inner trough 33 and an outer trough 37. The inner trough 33 has a fluid receiving interior 34 that receives the water exiting the fluid delivery conduit 29. As shown in FIG. 9, the inner trough 33 has a sidewall 34 that has a first upper edge portion 35 that is vertically higher than an opposing, second upper edge portion 36 so that the received fluid that enters the well 30 will spill in a predetermined direction (directed spill) into the outer trough 37 for draining into an overflow drain 38 and into a drainage tube 39 that carries the overflow fluid to a waste container or returns it to the original fluid loop 2. In a first embodiment, the second upper edge portion 36 can be formed or cut so that it is lower than the first upper edge portion 35. In another embodiment, the inner trough 33 can be angularly oriented within the outer trough 37 so that the second upper edge portion 36 is positioned further from the upper edge of the outer trough 37 than the first upper edge portion 35. In either embodiment, the water overflows the inner trough 33 through a gravity-induced crossflow. When fluid, such as water, samples are taken from within the well 30 as discussed below, these samples are taken from the fluid residing within the inner trough 33 at the time of sampling. A splash guard 31 can extend upward and form an upper portion of the outer trough that prevents water from spilling out of the outer trough 37. In an embodiment, the inner trough 33 can be securely attached to the lower surface of the outer trough 37. In another embodiment, the inner trough can be removably secured to an inner surface of the outer trough as shown in FIG. 9. The inner trough 33 can be lined with, or formed of, TEFLON or other materials, such as polyethylene (PE), to prevent endotoxins from binding to the inner surface of the inner trough 33.

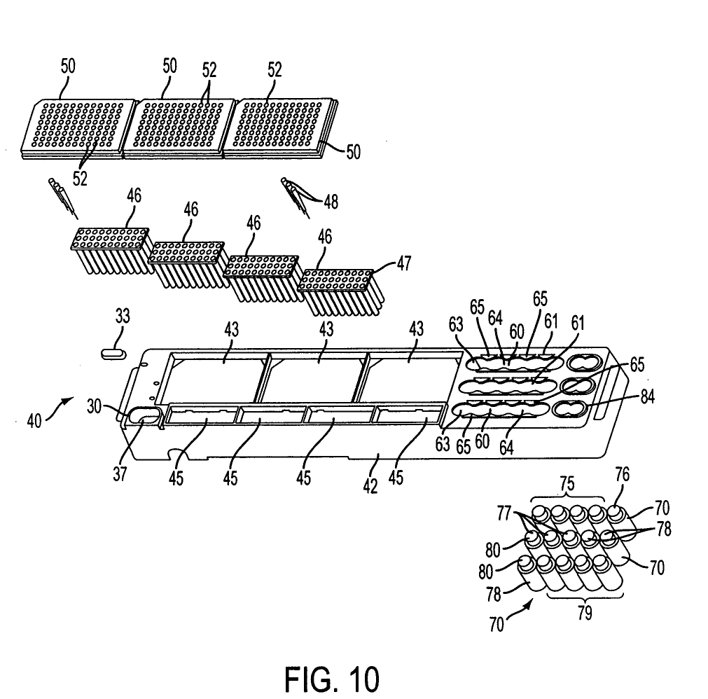

The cartridge housing 42 also has a plurality of openings for receiving other parts of the assembly 40 as shown in FIGS. 6 and 10. For example, the housing 42 includes at least one opening 43 for receiving at least one well plate 50. The illustrated cartridge housing 42, for example, includes at least three openings 43 that each receives a respective well plate 50. The well plates 50 can be snap-locked into the cartridge housing 42 so that they are removably secured to the cartridge housing 42. Resilient locking members carrying protrusions can extend through openings in the cartridge housing 42 to lock the well plates 50 to the cartridge housing 42. Other known removable securing members can be used to secure the well plates 50 to the cartridge housing 42.

Each well plate 50 includes a plurality of fluid receiving members, such as wells 52. The well plates 50 illustrated in FIG. 10 each includes ninety-six wells 52. However, well plates 50 can include greater or fewer wells 52 than the illustrated 96 wells. For example, the well plates 50 could each include between 100 and 400 wells per plate. As discussed below, the wells 52 receive fluids used in the water testing process.

The cartridge housing 42 also includes at least one opening 45 that can receive a respective well housing 46 for fluid carrying members, as shown in FIGS. 6 and 10. In the illustrated embodiment, the assembly 40 includes four openings 45 that each receives a respective tip well housing 46. Each tip well housing 46 includes a plurality of wells 47 that receive and hold new pipette tips 48 before they are used and contaminated pipette tips 48 after they have been used to deliver a fluid to one of the wells 52. These tip well housings 46 can each include about thirty wells 47. However, each tip well housing 46 can include greater or fewer than thirty wells 47. The number of wells 47 per cartridge housing 42 should provide a buffer of at least two empty rows of wells 47 between the used and the unused tips 48. It is possible to have none or only one empty row of empty wells 47 between the used and the unused tips 48. However, it is preferred that the cartridge housing 42 include at least two rows of empty wells between the used and unused tips 48. Each tip well housing 46 can be removably secured to the cartridge housing 42. The illustrated embodiment can carry about one hundred-five new and used tips 48.

The cartridge housing 42 also includes a plurality of rows 60 of vessel retention positions 61 that are arranged to receive fluid containing vessels 70 as shown in FIGS. 6 and 10. In the embodiment illustrated in FIG. 10, the cartridge housing 42 has three rows 60 of vessel retention positions 61 spaced from each other along the cartridge housing 42. In other embodiments, the cartridge housing 42 can have two rows 60, four rows 60 or greater than four rows 60 of vessel retention positions 61 for receiving fluid containing vessels 70. The number of rows 60 will depend on the number of fluid containing vessels 70 that are intended to be positioned within the cartridge housing 42.

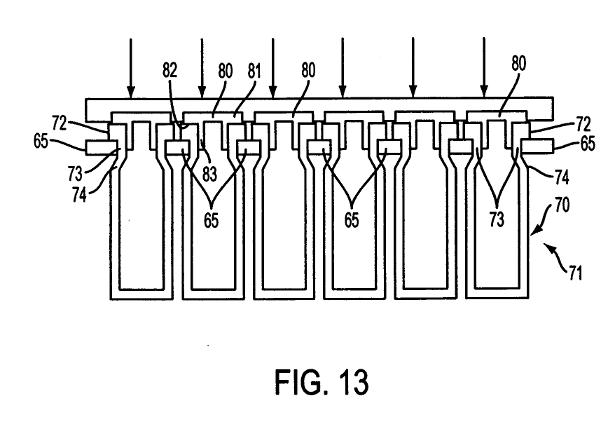

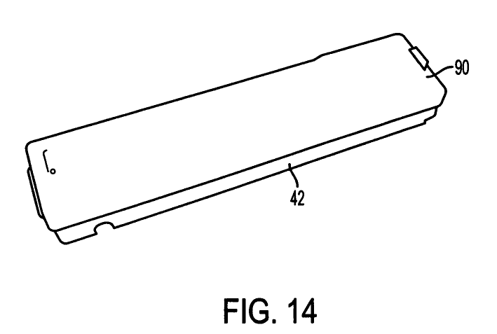

As shown in FIGS. 6 and 10, each row 60 of vessel retention positions 61 includes a plurality of openings 64 for receiving and supporting the fluid containing vessels 70. As shown in FIG. 14, each fluid containing vessel 70 has an elongated body 71. An upper end of each elongated body 71 has a radially protruding head 72 that is spaced from a radially protruding shoulder 73 by an elongated, vertically extending neck 74. In an embodiment, the fluid containing vessels 70 include vials. The terms “vessel” and “vial” does not limit the fluid containing vessels 70 to any particular shape or size. Instead, the vessels can be of any known shape or size that will fit within the rows 60 and can be engaged by securing members 65 to securely hold the vessels 70 with their respective rows 60. FIG. 13 shows an exemplary embodiment of the fluid containing vessels 70 according to the present invention. As shown in FIG. 13, the neck 74 has a smaller outer diameter when compared to the head 72 (above it) and the shoulder 73 (below it).

Each adjacent vessel retention position 61 includes a keyhole 63 through which the vessel 70 is introduced into the row 60 and a cooperating retention opening 64 in which a vessel 70 is securely retained. As shown in FIG. 10, a first end of each row 60 has an enlarged keyhole opening 63 into which a fluid containing vessel 70 can be introduced for then being positioned in the first opening 64 as shown in FIG. 10. Similarly, each adjacent vessel retention position 61 has its own associated larger keyhole opening 63 and smaller diameter retention opening 64. As a result, during production of the removable assembly 40, all of the vessels 70 may be inserted simultaneously through their respective keyholes 63 into the cartridge housing 42 and the entire cartridge housing 42 can be shifted horizontally to move the vessels 70 into position in their retention openings 64. The keyhole opening 63 has a greater diameter than the diameter of the fluid containing vessel 70. As a result, the fluid containing vessel 70 can be easily received and vertically positioned within a respective one of the rows 60. In an alternative embodiment, the openings 64 have a diameter that is substantially the same size as the diameter of the keyhole opening 63.

In either embodiment of the openings 64, securing members 65 extend into the openings 64 and engage the fluid containing vessels 70. As illustrated in FIGS. 10, 11 and 13, the securing members 65 included molded, projecting portions of the cartridge housing 42 that protrude into the open rows 60 and deflect sufficiently as the vessels 70 are being snap-fitted into the openings 64 so that the vessels 70 are removably received with the openings 64. The securing members 65 do not deflect enough to permit the removal of a vessel 70 as the positioning system 200 manipulates the vessel 70 and its cover 80. In another embodiment, the securing members 65 can be biased into engagement with the vessels 70 by a spring. Well known materials that will deflect enough to receive the vessel 70 and not break either the vessel 70 or the securing member 65 include well-known plastics.

As shown in FIG. 13, the ends of the securing members 65 are shaped to engage the outer surface of the neck 74 of the vessel 70 and abut against the head 72 and shoulder 73 when the vessels 70 are vertically moved within the cartridge housing 42. The positioning of the securing members 65 prevents vertical movement of the vessels 70 in both directions, while also preventing horizontal/lateral movement of the vials 70 within the rows 60. As understood, “horizontal” relates to the directions that are parallel with a plane in which an upper surface of the cartridge housing 42 lies that is parallel to the length of the rows 60. “Vertical”, on the other hand, is a direction that extends parallel to the height of the cartridge housing 42.

The fluid containing vessels 70 can carry a fluid used to test the fluid samples taken from within the inner trough 33 (sample well), contained within the well 30. In an embodiment illustrated in FIG. 10, a first set of vials 75 carry an enzyme for delivering to the wells 52. In an embodiment, four vials 75 can each have an internal fluid capacity of about 5 cc to 10 cc and carry a total fill volume of about 1.2 cc or greater of an enzyme. The enzymes that can be contained in the vials 76 include those discussed herein including “PytoGene™”. At least one vial 76 can include an endotoxin. In an embodiment, the vial 76 has an internal volume of about 10 cc and contains about 7 cc of the endotoxin. Endotoxins carried by vial 76 can include E. coli. The fluid containing vessels 70 can also include three vials 77 for carrying a substrate. Each of the three illustrated vials 77 has an internal capacity of about 10 cc. The three vials 77 have carry a total volume of about 6 cc of a preferred substrate. Substrates useable with the present invention include any known chromogenic or fluorogenic substrate that can identify the presence of an endotoxin. An additional set of vials 78 can carry any conventional buffer including those discussed herein. Each of the illustrated vials 78 has an internal capacity of about 10 cc and they carry a total combined fill volume of about to 5 cc of a buffer. Another set of vials 79 can carry clean control water. In the illustrated embodiment, the assembly 40 includes four 10 cc vials that hold a total of about 11.5 cc of water. In any of the above embodiments, the vials can have a greater internal volume than the volume mentioned above. Similarly, the fluid containing vessels 70 can be filled to include more or less of their respective fluids. Additionally, the number of vials carrying each liquid can be greater or less than mentioned above. Furthermore, the buffer and substrate may be combined to form one liquid reagent. Similarly, the buffer, substrate and recombinant enzyme may also be combined and lyophilized to form one freeze dried reagent.

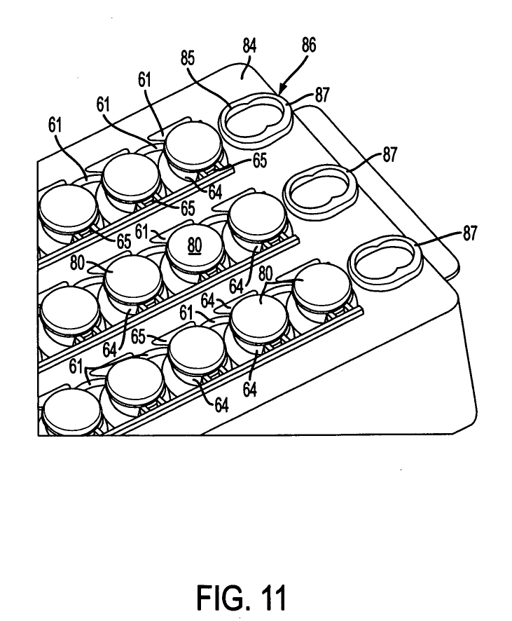

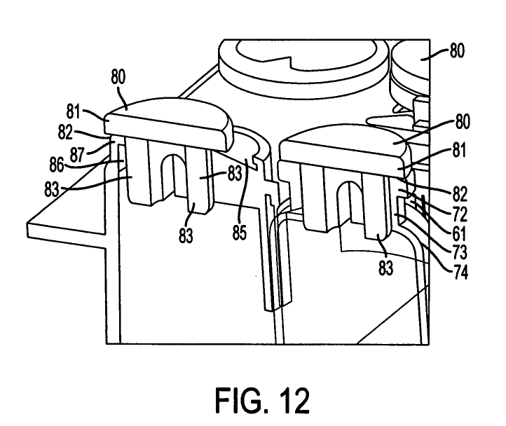

The cartridge housing 42 also includes a plurality of slotted openings 84 for receiving covers 80 from the vessels 70, as illustrated in FIGS. 10 and 12, while the vessel 70 is being accessed and fluids within the vessel 70 are being taken. The covers 80 include a flange 81 with a lower surface 82 and a plug portion 83 for positioning within an opening of one of the vessels 70. Each opening 84 includes a keyhole 85 with a first diameter and a retaining hole 86 with a second diameter. As seen in FIG. 11, the diameter of the keyhole 85 is greater than the diameter of the retaining hole 86. As a result, the cover 80 can be introduced into the keyhole 85 vertically, as discussed below, and then slid horizontally into the retaining hole 86. The retaining hole 86 receives the cover 80 as shown in FIG. 12. An upper flange 87 extending around a portion of the retaining hole 86 engages the lower, under surface 82 of the flange 81 and supports the cover 80 within the retaining hole 86. As seen in FIG. 12, the under surface 82 of the flange 81 is the only surface contacted by a portion of the cartridge housing 42 (Flange 87). The plug portion 83 that extends into the vessel 70 does not come into contact with the cartridge housing 42 as it is being introduced into the keyhole 85 and slid into the retaining hole 86. As a result, any fluid or materials on the plug portion 83 of the cover 80 do not come in contact with and contaminate the cartridge housing 42. Similarly, the covers 80 of different vessels 70 will not be contaminated by the cartridge housing 42.

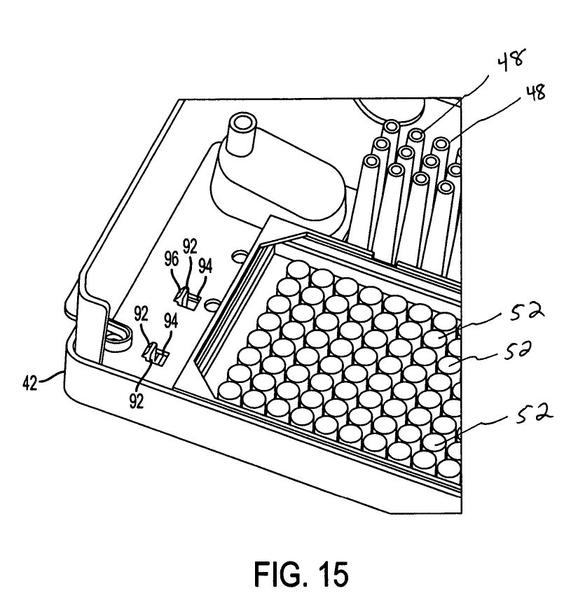

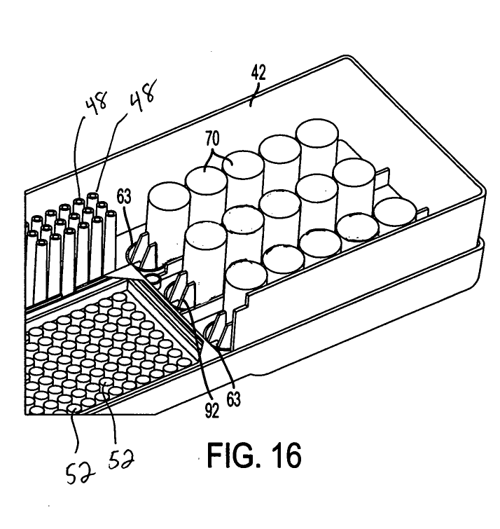

The replaceable cartridge assembly 40 can also include a cover 90 that is removably secured over the cartridge housing 42 (FIG. 14). In an embodiment, the cover 90 could include a rubber serum stopper or a lyophilization stopper. The cover 90 protects the contents of the original or a replacement cartridge assembly 40 prior to the cartridge assembly 40 being placed within the housing 11. The cover 90 includes posts 92 (FIGS. 15 and 16) that extend into openings 94 in the cartridge housing 42. Each post 92 includes at least one securing protrusion 96 that is received within an opening 94 in the cartridge housing 42 and/or the keyhole opening 63 so that the post 92 and cover 90 are secured to the cartridge housing 42 until the assembly 40 is ready to placed into the housing 11. Each securing protrusion 96 can include at least one tooth or other member that can releasably engage with the cartridge housing 42 to prevent the unintentional removal of the cover 90 from the cartridge housing 42. Prior to, or after insertion of the assembly 40 into the housing 11, the cover 90 can be removed from the cartridge housing 42 by deflecting the posts 92 and their respective teeth 96 away from engagement with the cartridge housing 42. Prior to being removed, the posts 92 and their respective teeth 96 can abut against one or more of the vessels 70 and secure them against movement relative to the cartridge housing 42. Similarly, a plurality of the posts 92 and their respective securing protrusions 96 can also secure the well plates 50 and the tip well housings 46 against movement when they are covered by the attached cover 90.

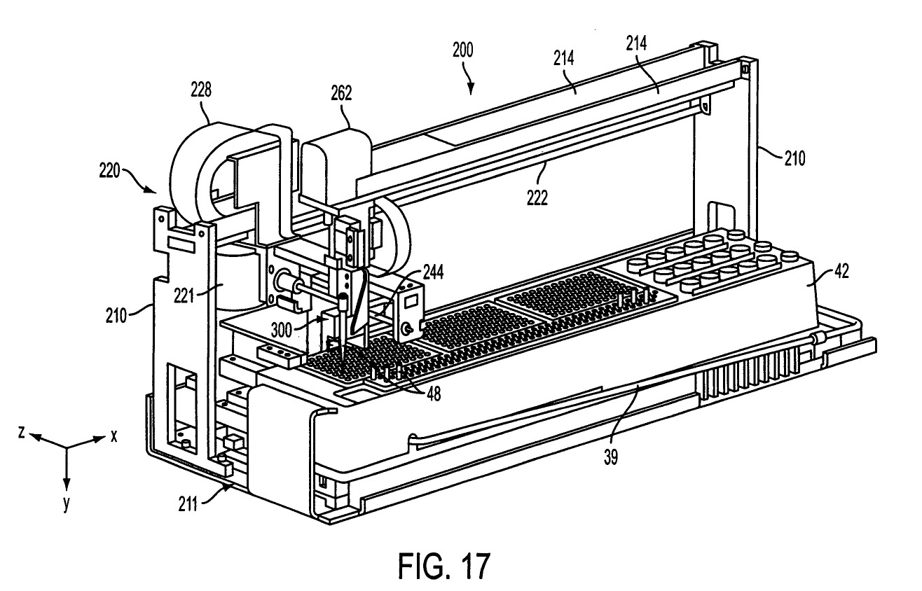

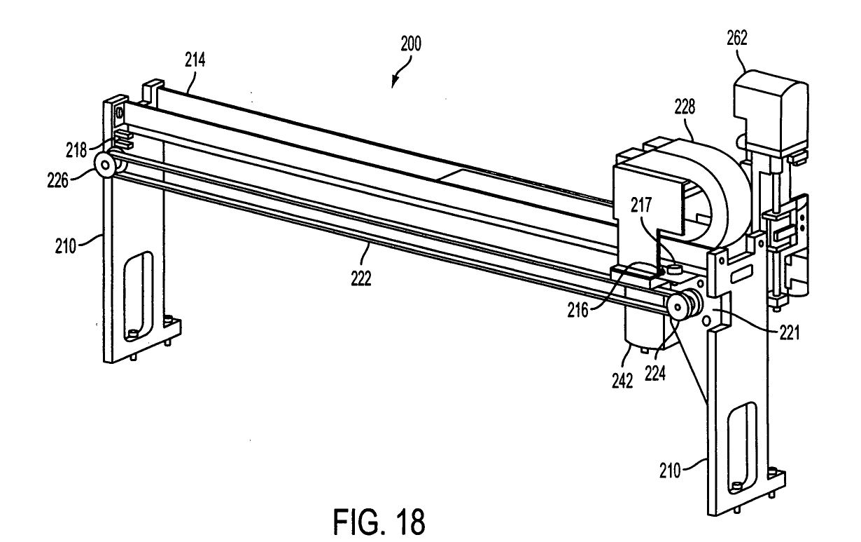

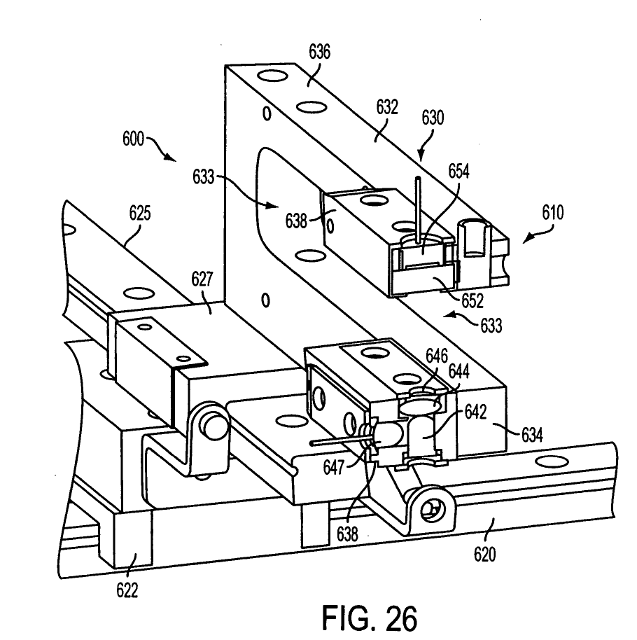

The apparatus 10 also includes a motorized positioning system 200 (FIG. 17) positioned within housing 11. In an embodiment, the positioning system 200 can include the illustrated motorized robotic arm. The motorized positioning system 200 carries and manipulates an integrated, articulatable head assembly 300 along X, Y and Z axes as shown in FIG. 18. As discussed below, the head assembly 300 can remove the covers 80 from the vessels 70, retrieve tips 48, obtain fluids from within the vessels 70 and the well 30, deliver the fluids to the wells 52 in well plates 50 and test for the presence of endotoxins in the fluid containing wells 52. The positioning system 200 can also position a fluorescent detection assembly 610 and/or a fiber optic fluorescent reader (FIGS. 19A and 26) (carried by the head assembly 300 or separate from the head assembly 300) over the fluid containing wells 52 in the well plates 50 and move the head assembly 300 so that it removes a cover 80 from an opening 84 and returns it to, and in, its respective vessel 70.

As shown in FIGS. 17 and 18, the positioning system 200 includes vertical mounts 210 that are secured to mounting plate 211 positioned in the housing 11. First and second linear guiding and supporting rails 214 extend between the vertical mounts in a direction along (parallel to) the X-axis to provide support and stiffness to the positioning system. During the operation of the assembly 10, the head assembly 300 can travel along the length of the rails 214 when an X-axis drive system 215 including a linear motion motor system 220 and a plurality of travel sensors 216 is operated. The travel sensors 216 limit the length of travel of the head assembly 300 along the rails 214. The travel sensors discussed herein can be sensors that are activated by contact, by breaking a light beam emitted by the sensors or by causing motion within each sensors predetermined field of view. The travel sensors 216 can include a home sensor 217 and a limit sensor 218. The travel sensors 216 can be any known motion limiting sensor that determines the linear movement of a member and controls a motor accordingly.

In the illustrated embodiment, the linear motion motor system 220 includes a housing 221, an endless toothed belt 222, a driven toothed pulley 224 and a follower pulley 226. The driven toothed gear 224 is driven and powered by a conventional rotary stepper motor (not shown) within housing 221. As will be understood, the teeth of the pulleys 224, 226 engage the teeth of the belt 222 in order to drive the head assembly 300 along the rails 214. When the head assembly 300 activates either travel sensor 216, the operation of the motor can be stopped and the direction of motion of the motor and the driven pulley 224 can be reversed so that the head assembly 300 travels in a direction away from the activated sensor 216. In other embodiments (not shown), the pulley 224 can be driven by a conventional linear variable reluctance motor or a powered rack and pinion. The positioning system 200 can also include a cable guide 228 as known in the art. Also, the positioning system 200 can have a half-stepping resolution of about 0.006 inch.

The positioning system 200 can also move the head assembly along the Y-axis, illustrated in FIG. 17. Y-axis motion is created by the operation of a Y-axis drive system 240 including a linear motor system 242 and a plurality of motion limiting sensors 247 (FIG. 19B). The linear motor system 242 includes a rotary motor 243 and a lead screw 244 that extends at least the entire Y-axis travel distance. The rotary motor 243 drives the lead screw 244 as it operates. Any other known linear motion system, including those discussed above, can be used Y-axis drive system 240.

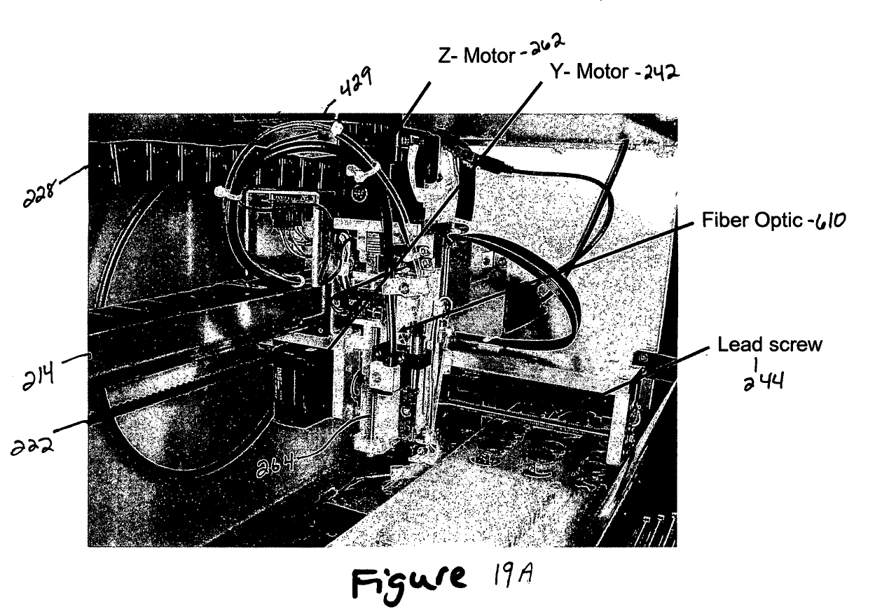

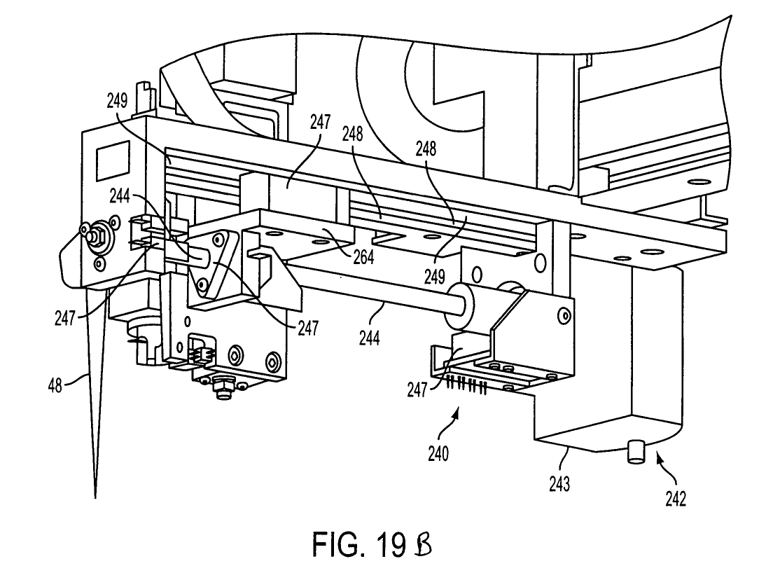

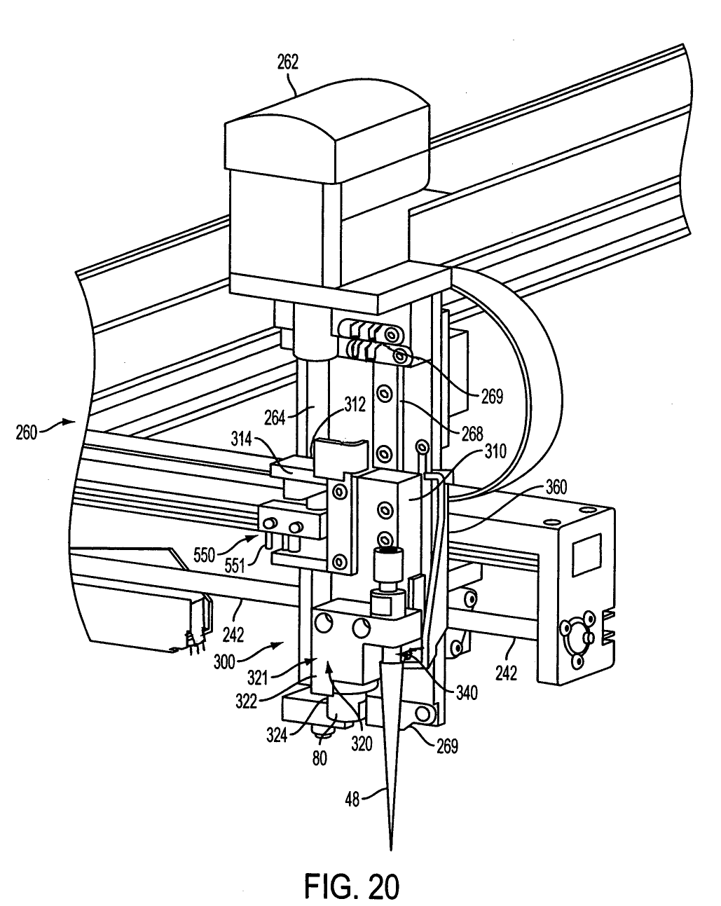

As illustrated in FIG. 19B, the head assembly 300 is operatively secured to a mounting platform 246 having an opening 247 through which the lead screw 244 extends. An internal surface of the opening 247 includes threads that mesh with and operatively engage the lead screw 244 so that the head assembly 300 moves along the length of the lead screw 244 into a predetermined position as the lead screw 244 rotates relative to the rails 214. The platform 246 is secured to a support bracket 247 that includes projections that travel within grooved tracks 248 of a support member 249 secured to the housing 221 so that head assembly 300 secured to the platform 246 moves with the housing 221. As illustrated in FIG. 20B, the support member 249 can include one or more elongated, grooved rails extending below a slide 241. The Y-axis drive system 240 has a half-stepping resolution of about 0.0005 inch.

As shown in FIGS. 19A and 20, the positioning system 200 also includes a Z-axis drive system 260 for moving the head assembly 300 along the vertical Z-axis. The Z-axis drive system 260 includes a rotary motor 262 and lead screw 264 that cooperate to drive a sliding member 310 of the head assembly 300 along a grooved linear slide 268. The Z-axis drive system 260 operates in a substantially similar manner as the Y-axis drive system 240. As show in FIG. 20, the lead screw 264 extends through a threaded opening 312 in a portion 314 of the sliding member 310. As the rotary motor 262 turns, the lead screw 264 is driven in one of the two rotary directions. This rotation of the lead screw 264 causes the sliding member 310 and the head assembly 300 to move vertically either in the direction of the cartridge housing 42 or away from the cartridge housing 42. Travel limiting sensors 269 prevent the sliding member 310 from moving beyond predetermined locations along the lead screw 264. As with the sensors used with the Y-axis system, the travel limiting sensors 269 can be any known sensor including those discussed above with respect to the X-axis drive system 215.

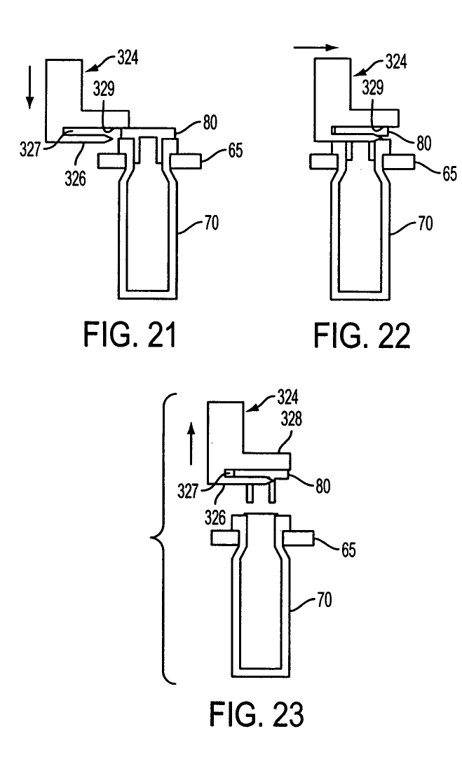

In addition to the sliding member 310, the head assembly 300 also includes a system 320 for engaging and removing the covers 80 from the vessels 70, as shown in FIGS. 20-23. The system 320 includes a housing 322 secured to the sliding member 310. As illustrated, the housing 322 can be secured to the sliding member 310 proximate the portion 314 that threadably receives the lead screw 264. The housing 322 has a lower portion that forms a lifting fork 324 for removing the covers 80 from the vessels 70, positioning the covers 80 in the openings 84 and returning the covers 80 to their respective vessels 70. In the illustrated embodiment, the lifting fork 324 includes a pair of spaced fork members 326 that have tapered forward ends for being introduced under a cover 80 (FIG. 21). The fork members 326 are spaced from each other by a gap that is sized to receive the plug portion 83 of the cover 80. The gap between the fork members 326 is sized greater than the diameter of the plug portion 83 so that the gap receives the plug portion 83 without engaging and being contaminated by the plug portion 83. The lifting fork 324 includes an upper retaining member 328 that extends over the fork members 326 as illustrated so that a cover receiving space 327 is formed between the lifting forks and the lower surface 329 of the retaining member 328. The cover member 328 holds the cover 80 of the vessel 30 within the fork members 326. As a result, the lifting fork 324 is able to manipulate the cover 80 as it removes it from the vessel 70, places it within an opening 84, retrieves it from within hole 84 and returns the cover 80 to the vessel 70.

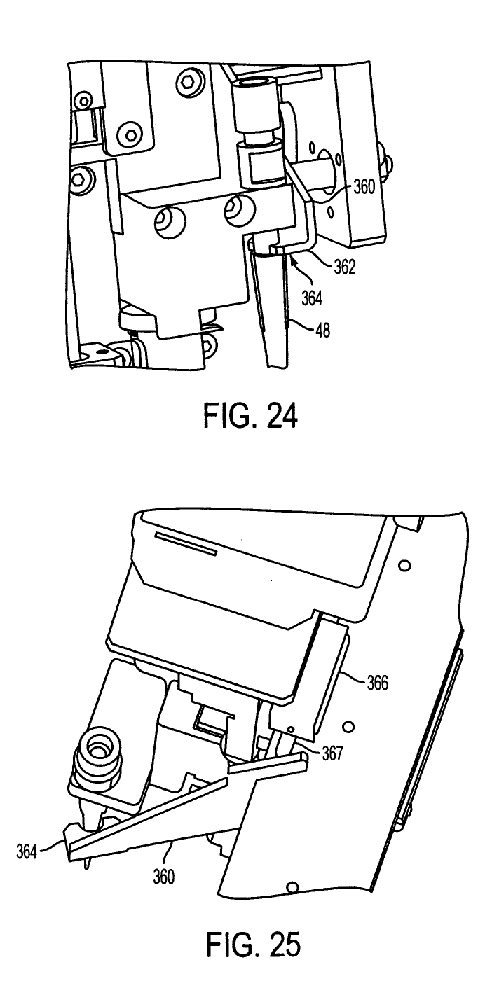

As illustrated in FIGS. 20, 24 and 25, the head assembly 300 further includes a tip coupling member 340 and a tip ejector 360. The tip coupling member 340 can be formed as a portion of the housing 322, as illustrated, or it can be separate from the housing 322. In either embodiment, the tip coupling member 340 is vertically moveable along the Z-axis. In the illustrated embodiment, the tip coupling member 340 includes an elongated, tapered member that is sized to be introduced into the hollow interior of a tip 48 as the tip coupling member 340 moves in a downward direction into engagement with one of the unused tips 48. The tip coupling member 340 is introduced into and positioned within a tip 48 as the sliding member 310 and housing 322 move vertically downward toward the tips 48. The tip coupling member 340 will frictionally engage the inner surface of a hollow tip 48 and remove it from its tip well 46 as it moves vertically upward away from the cartridge housing 42.

In order to separate the tip 48 from the tip coupling member 340 and eject the used tip 48 into a tip well 46, a lower surface 362 of a forked portion 364 of the ejector 360 engages the used tip 48 that has been positioned within a tip well 46. The ejector 360 is brought into engagement with the tip 48 to be removed by a solenoid switch 366 that activates a plunger or piston rod 367 that is driven into contact with a portion of the ejector 360 (FIG. 25). The rod 367 can be driven by any known drive source. After the rod 367 contacts the ejector 360, the ejector 360 is rotated into engagement with the held tip 48. While the ejector 360 remains stationary and the lower surface 362 is engaged with the tip 48, the sliding member 310 and housing 322 are moved vertically upward in a direction away from the forked portion 364 of the ejector 360. The forked portion 364 prevents the tip 48 from moving as the tip coupling member 340 is raised away from the respective tip well 46. As a result, the tip 48 is separated from the tip coupling member 340 and left in a respective tip well 46.

The head assembly 300 also includes a position sensing system 550 (FIG. 20) for detecting the position of a cover 80 with respect to its vessel 70 and the position of a tip 48 with respect to a respective tip well 46. The position sensing system 550 is particularly useful for determining the position of the cover 80 after removal from opening 84 or the position of a tip 48 after it has been used. The sensing system 550 includes a sensor 551 that can determine when the sliding member 310 is encountering resistance to its motion along the Y-axis in the direction of the cartridge housing 42 as a result of completing a vertical throw and either picking up or returning a cover 80 or tip 48. When the sensor 551 has determined that the sliding member 310 is encountering resistance and that the cover 80 has been returned to the vessel 70, the sensor 551 causes a switch to turn off the Y-axis drive motor.

In a first embodiment, the sensor 551 can be positioned proximate the portion 314 of the sliding member 310 that receives lead screw 264. As a result, the sensor 551 will be engaged by the portion 314 as the portion 314 deflects in response to the stopping of the motion of the forward portion of the sliding member 310 and the continued rotation of the lead screw 264. Alternatively, the sensor 551 activates a switch that stops the operation of the Y-axis motor when a spring loaded member is deflected into contact with the sensor 551 or the spring loaded member is deflect across a beam or into the vision of the sensor 551. When the spring loaded member contacts the sensor 551 in response to the stopping of the sliding member 310, the assembly 10 understands that the sliding member 310 has completed a vertical throw and either picked up or returned a cover 80 or tip 48. This length of the vertical distance traveled by the sliding member 310 can also provide information to the processor and control system of the assembly 10 regarding the height at which horizontal motion of head assembly 300 takes place, thereby making the motion of the assembly more efficient.

As shown in FIG. 26, the assembly 10 can also include a system 600 that provides a chromogenic or flurogenic detection scheme for determining the presence of trace levels of endotoxins within the tested water from well 30. The system 600 includes a detection assembly 610 that moves along the X-axis and the Y-axis. The detection assembly 610 includes a first grooved rail 620 that extends along the X-axis and a second grooved rail 625 that extends along the Y-axis. A detector head 630 is secured to the rails 620, 625 by brackets 622 and 627, respectively. The brackets 622 and 627 are secured to each other by mounting plates 628. The detection assembly 610 moves along the X-axis and the Y-axis via the operation of the X-axis and Y-axis drive systems 215, 240 used to drive the head assembly 300. The detector head 630 can be operatively secured to the positioning system 200 so that it moves along the X-axis and Y-axis when the head assembly 300 moves along these axes or when the control processor of the assembly 10 activates the positioning system 200 to move the detection assembly without regard for the position of the head assembly 300.

As shown in FIG. 26, the detector head 630 includes a U-shaped member 632 that has a recess 633 in which the well plates 50 are received. As can be seen, a lower arm 634 of the U-shaped member 632 is positioned beneath the well plates 50 as the detector head moves along the cartridge housing 42. An upper arm 636 of the U-shaped member 632 extends over the well plates 50 as the detector head moves. The lower arm 634 has a plurality of passageways 640 that carry an LED 642 and a lens 644 positioned above the LED 642. The lens 644 covers the aperture 646 at the upper end of the passageway 640. A feedback detector 647 is positioned within a passageway 648 that extends within the lower arm 634 at an angle to the LED 642. The feedback detector 647 provides information to the operating system of the assembly 10. The upper arm 636 includes a slot carrying a conventional filter 652, such as a solid state detector (photodiode) for absorbance assays, and a conventional photomultiplier detector 654 for fluorescence assays, such as those used in the industry, for example by Bio-Tek Instruments. The upper arm 636 also includes apertures within its outer surfaces for receiving light transmissions as is understood in the art. In an alternative embodiment, the upper arm 636 does not include the filter 652. Also, the detector head 630 can have any shape that allows a first portion to extend under the well plates 50 and another portion to extend above the well plates 50. In another embodiment, the head assembly 300 carries a fiberoptic fluorescent reader that will move over the well plates 50 and take the appropriate readings as the head assembly 300 moves over the cartridge housing (See FIG. 17). In each of the above-discussed embodiments, light from the LED can be transmitted through a transparent and/or translucent lower surface of each well 52. Also, light can be delivered to the LED and data can be transmitted from the detectors using fiberoptics. The illustrated U-shaped assembly that reads through the microplate wells is a preferred embodiment for assays using absorbance detection, including endotoxin detection and more commonplace assays such as enzyme-linked immunosorbant assays (ELISAs). This detector head 630 enables the use of the head assembly 300 for pipetting and moving materials as well as coupling light through a microplate well 52 with the detachable optics assembly 638. The use of a single bundled fiber optic (FIG. 20) on the head assembly 300 can be used in a preferred embodiment with fluorescence assays.

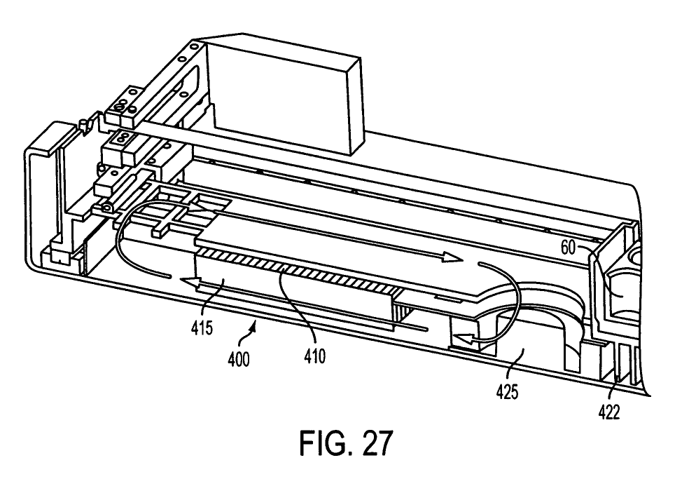

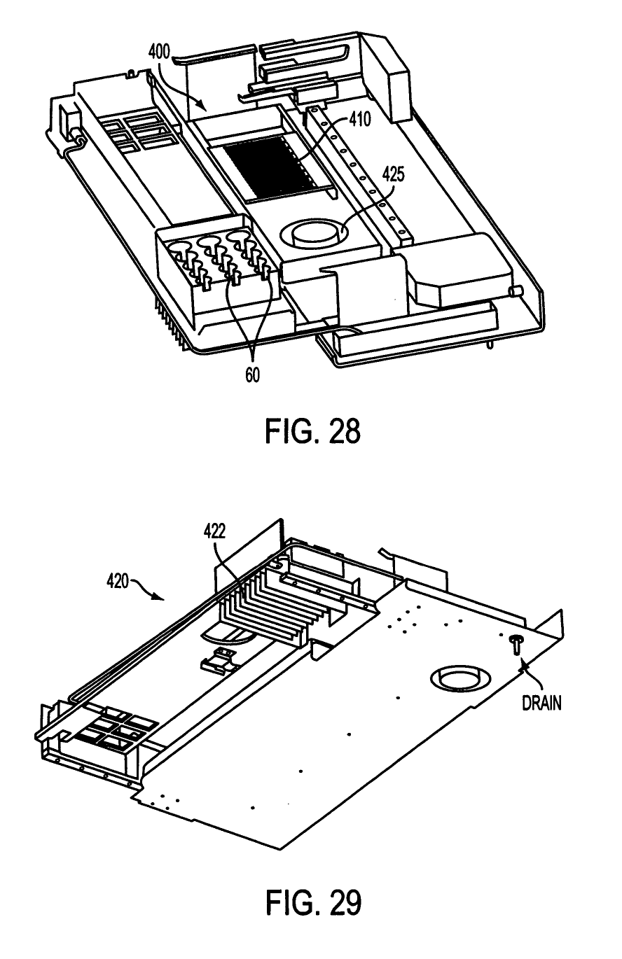

As shown in FIGS. 27-29, the assembly 10 can also include a heating system 400 for warming the fluid well plates 50 and a cooling system 420 for maintaining cool temperatures around the vessels 70. The heating system 400 can include a heating element 410 that is positioned under the well plates 50 when the removable and replaceable cartridge housing 42 is positioned within housing 11. In the illustrated embodiment, the heating element 410 includes a resistive heating element. Alternative known heating elements may also be used. A heat sink 415 can line the exterior vertical walls of the heating element to prevent heat from being radiated along the X-axis or the Y-axis. The cooling system 420 can include a source of cold air or refrigeration that cooperates with a plurality of cooling fins 422 on a lower surface of the cartridge housing 42 beneath the rows 60 carrying the vessels 70. The cooling system 420 uses an electronic cooling device. In an embodiment, the cooling device includes a PELTIER thermoelectric cooler. A blower fan 425 is used to draw air into the housing 11 and across the heat sink 422 in order to remove heat from the cooling block within the cartridge housing 42. An additional fan 429 positioned in the top of the internal housing transfers air from the upper chamber (electronics bay) to the low chamber (robot housing) through a high-efficiency (HEPA-type) filter that reduces the introduction of airborne contaminants into the chamber carrying the positioning system 200. The fan 429 also creates positive pressure in the housing 11.

The assembly 10 further includes a syringe pump 700 that is in fluid connection with the head assembly 300 (FIG. 4). The head assembly 300 includes a vacuum port that creates a vacuum in the tips 48 and draws fluids from well 30 and vessels 70 into the tips 48 in response to the intake stroke of the piston of the syringe pump 700. As the syringe pump 700 returns to rest, the vacuum within the carried tip 48 is released and the fluid is expelled into its respective well 52 in one of the well plates 50.

In operation, the assembly 10 will receive and test a water sample from the loop 2 as previously discussed. In the manner discussed above, water from the loop 2 enters the flow path 16 and passes through the fluid delivery system 20 and into the well 30 in the manner discussed above. The positioning system 200 moves the head assembly along the X-axis and/or Y-axis until the tip coupling member 340 is positioned over a tip 48 within a tip well 46. The sliding member 310 is then moved along the Z-axis until it engages a tip 48 and the sensor 551 is activated. When this occurs, the stroke of the sliding member 310 is reversed so that the tip 48 is removed from the tip well 46. The processor and control system of the apparatus 10 then cause the positioning system 200 to locate the carried tip 48 over the inner trough 33 of the well 30. Once the tip 48 is positioned over the trough 33, the sliding member 310 is then driven vertically downward until the tip 48 engages the fluid within the inner trough 33. The syringe pump 700 is then activated so that fluid to be tested is drawn up from the inner trough 33 into the tip 48.

The fluid carry tip 48 is then moved by the positioning system 200 until it is positioned over a well 52 of the well plates 50. The fluid carrying tip 48 is then driven toward the well 52 by the Z-axis drive system 260. Upon reaching a predetermined height, an amount of the carried fluid for testing is released into a first well 52 by the operation of the syringe pump 700 as discussed above. The method of the present invention can include duplicating each test in a plurality of separate wells 52. As a result, before the fluid within the tip 48 is released, the fluid carrying tip 48 can be moved to a second well 52 and the step of releasing the carried fluid into a well 52 can be repeated. After the carried fluid is released into the two wells 52, the used tip 48 is located over an empty tip well 47 by the positioning system 200. The empty tip well 47 is preferably spaced from the unused tips 48 by a space comprising at least one row of tip wells 47, as discussed above. Once the used tip 48 is positioned within the tip well 47, the sensing system 550 determines when the tip 48 has been fully inserted into its well 47 as discussed above and the tip ejector 360 separates the used tip 48 from the tip coupling member 340 in the manner discussed above. Then, the head assembly 300 is moved by the positioning system 200 along the cartridge frame 42 toward the vessels 70.

Upon reaching the vessels 70, the lifting fork 324 is moved vertically along the Z-axis into position proximate a cover 80 of the vials 79 carrying the control water (FIG. 21). The lifting fork 324 is then moved horizontally so that the fork members 326 are positioned between the underside 82 of the cover 80 and the head 72 of the vessel 70 (FIG. 22). Once the cover 80 is received and positioned in the cover receiving space 327, the cover 80 is removed from its vessel 70 (FIG. 23) by lifting the lifting fork 324 vertically away from the vessel 70. The positioning system 200 then places the cover 80 over the keyhole 85 of the opening 84 and lowers the cover 80 to the opening 84 so that the plug 83 is positioned within the keyhole 85. The introduced cover 80 slides horizontally into its retaining hole 86 in response to the movement of the positioning system 200. The lifting fork 324 separates from the retained cover 80 and the head assembly 300 returns to the tip wells 46.

Upon returning to the tip wells 46, the tip coupling member 340 obtains another tip 48 in the manner discussed above and moves this tip 48 into position over the open vial 79 of the control water. The positioning system 200 then moves the sliding member 310 along the Z-axis and the carried tip 48 into the open vessel 79. The syringe pump 700 then operates to withdraw the control water from the vial 79 and into the tip 48. The control water carrying tip 48 moves into position over the well plates 50 as discussed above with respect to the water from trough 33 and releases the control water into at least two wells 52. In a preferred embodiment, the control water is released into at least four wells 52. The positioning system 10 then locates the used tip 48 over one of the empty tip wells 47 and the used tip 48 is ejected into the empty tip well 47 as previously discussed.

After the used tip 48 that carried the control water is positioned within the tip well 47, the positioning system 200 then positions the lifting fork 324 proximate the cover 80 located in the hole 86. The sliding member 310 moves along the Z-axis and brings the lifting fork 324 to the level of the cover 80. The positioning system 200 causes the lifting fork 234 to engage the cover 80 and move the cover into the keyhole 85, where the cover is then removed from the opening 84 and returned to its vessel 70. After the lifting fork 324 has returned the cover 80 to its vial 79, the head assembly 300 returns to the vessels 70 in preparation for removing the cover 80 from another of the vessels 70. The steps of removing a cover 80, securely placing the cover 80 within the opening 84, obtaining a tip 48 from a tip well 47, obtaining a fluid from the open vessel 70, introducing the obtained fluid into appropriate wells 52, ejecting the used tip 48 and returning the removed cover 80 to the open vessel 70 are done for each of the other fluids in the vials 70 in the manner discussed above. However, the endotoxin from vial 76 is only positioned in one of the wells containing the control water if only three wells 52 are being used in the test. In an embodiment in which the test is being duplicated and at least six wells 52 are being used, the endotoxin is introduced into two, or half, of the wells 52 containing the control water.

In an embodiment of the method, the system 320 for removing and positioning the covers 80 removes the cover 80 from one of the substrate vials 77 and the buffer vials 78 before obtaining an unused tip 48 so that the substrate vial 77 and the buffer vial are open at the same time. In this embodiment, the same tip 48 can be used to obtain and deliver the substrate and the buffer to each of the wells 52 containing the water to be tested and each of the wells 52 containing the control water. A different tip 48 from that used to deliver any of the other fluids receives and delivers the enzyme from vessel 75 to the wells 52 containing the water to be tested and the wells 52 containing the control water. The fluids from the vials 70 and the fluid to be tested, such as water, can be introduced into the wells 52 in any order. The order of delivering fluids to the wells 52 discussed above is not limiting on the method of the present invention.

Once the fluids from the vials 75-79 and the water to be tested have been positioned in their appropriate wells 52, the detection system 600 including the detection assembly 610 and/or the fluorescent reader positioned on the head assembly 300 are passed over the fluid containing wells 52. The detection system 600 determines either the optical density of the fluid containing wells 52 in the chromogenic or turbidimetric methods, or the relative fluorescent intensity of the fluid containing wells 52 in the fluorescent method. The detection system 600 then compares the results from its scan of the fluid containing wells and identifies if an endotoxin is present in the tested water.

All patents, patent applications, and references cited in this disclosure are expressly incorporated herein by reference.

The above discussions do not limit the invention. Although the disclosure describes and illustrates preferred embodiments of the invention, it is to be understood that the invention is not limited to these particular embodiments. Many variations and modifications will now occur to those skilled in the art.

1. An apparatus for positioning within a fluid system line for performing on-line testing of fluid within the line for the presence of an endotoxin, said apparatus comprising:
a fluid sampling system for obtaining a fluid sample from the fluid system line;
an assembly including a fluid receiving member into which said fluid sample and an agent are introduced; and
a detection system for determining the presence of an endotoxin within said fluid receiving member.

2. The apparatus of claim 1 wherein said agent includes a chromogenic substrate and/or a reagent.

3. The apparatus of claim 1 wherein said fluid sampling system contains a solenoid valve that operates to control the flow of fluid into said fluid sampling system.

4. The apparatus of claim 3 wherein said fluid sampling system further comprises a fluid storage container downstream of said solenoid valve for receiving the fluid sample from the fluid system line when said solenoid valve is open.

5. The apparatus of claim 4 further comprising a fluid conduit for delivering the fluid sample from the fluid storage container to a flow well.

6. The apparatus of claim 5 wherein a terminal end of said fluid conduit is spaced from said flow well and said flow well forms a portion of said assembly.

7. The apparatus of claim 5 wherein said flow well comprises an inner trough and an outer trough that surrounds said inner trough and receives fluid overflow from said inner trough.

8. The apparatus of claim 7 further comprising a fluid line extending from said flow well to carry the overflowed fluid in said outer trough away from said flow well.

9. The apparatus of claim 7 wherein said flow well further comprises a splash guard.

10. The apparatus of claim 1 wherein said assembly is removably secured within a housing of the apparatus.

11. The apparatus of claim 1 wherein the assembly includes a cartridge assembly removably secured within a housing of said apparatus.

12. The apparatus of claim 11 wherein said cartridge assembly further comprises a plurality of removable plates.

13. The apparatus of claim 11 wherein said cartridge assembly includes a tip well housing comprising a plurality of wells for receiving pipette tips.

14. The apparatus of claim 11 wherein said cartridge assembly includes at least one well for receiving at least a portion of the fluid sample.

15. The apparatus of claim 11 wherein the cartridge assembly further comprises a plurality of vessel retention positions.

16. The apparatus of claim of claim 15 wherein each vessel retention position includes a first opening into which a vessel is inserted and a second opening that is open to the first opening for slidably receiving the vessel from the first opening, wherein the first opening is larger than said second opening.

17. The apparatus of claim 15 wherein said second opening includes securing members for engaging an inserted vessel.

18. The apparatus of claim 15 wherein said cartridge assembly further comprises a plurality of cover receiving openings, each for receiving a cover of a respective vessel.

19. The apparatus of claim 1 further comprising a motorized positioning system including a powered arm and a head assembly for performing a plurality of functions.

20. The apparatus of claim 19 wherein said head assembly further comprises a member for engaging and retaining fluid carrying members positioned within wells in said assembly.

21. The apparatus of claim 20 wherein said head assembly further comprises an ejector for removing spent fluid carrying members from the head assembly.

22. The apparatus of claim 19 wherein said positioning system includes a plurality of travel sensors for limiting the movement of said head assembly in at least one plane.

23. The apparatus of claim 1 further comprising a detector head for determining the presence of an endotoxin within the fluid sample in said fluid receiving member.

24. The apparatus of claim 23 wherein said detector head includes a light source and a detector.

25. The apparatus of claim 24 wherein said light source is an LED.

26. The apparatus of claim 1 further comprising a heating system for providing heat to the assembly and a cooling system for maintaining cool temperatures around vessels within said assembly.

27. An apparatus for performing on-line testing of a fluid in a fluid system line to determine the presence of endotoxin, said apparatus comprising:
a housing;
a fluid sampling system for positioning in the fluid system line such that fluid from said fluid system line enters into said fluid sampling system in response to a change in pressure within said fluid sampling system;
a fluid flow well positioned downstream of said fluid sampling system for receiving the fluid therefrom;
a removable assembly comprising a plurality of wells for holding at least one fluid carrying members, a plurality of wells for holding a portion of a sample of the fluid received from said fluid sampling system and at least one fluid vessel retention position;
a positioning system including a head assembly for retrieving and moving said at least one fluid carrying member relative to said removable assembly,
a source of negative pressure operatively connected in fluid communication with said head assembly for drawing the fluid from the fluid flow well and endotoxin identifying agents into respective fluid carrying members; and
a detector system for determining if an endotoxin is present within the fluid sample.

28. The apparatus of claim 27 wherein said endotoxin identifying agent includes a chromogenic substrate and/or a reagent.

29. The apparatus of claim 27 wherein said fluid sampling system contains a solenoid valve that operates to create said pressure change and control the flow of the fluid sample into said fluid sampling system.

30. The apparatus of claim 29 wherein said fluid sampling system further comprises a fluid holding member downstream of said solenoid valve for receiving the fluid from the fluid system when said solenoid valve is open.

31. The apparatus of claim 30 further comprising a fluid conduit for delivering the fluid from the fluid holding member to said fluid flow well.

32. The apparatus of claim 31 wherein a terminal end of said fluid conduit is spaced from said flow well and said flow well forms a portion of said removable assembly.

33. The apparatus of claim 31 wherein said flow well comprises an inner trough and an outer trough that surrounds said inner trough, said outer trough receives fluid overflow from said inner trough.

34. The apparatus of claim 33 further comprising a fluid line extending from said flow well to carry the overflowed fluid in said outer trough away from said flow well.

35. The apparatus of claim 33 wherein said flow well further comprises a splash guard.

36. The apparatus of claim 27 wherein the removable assembly includes a cartridge assembly removably secured within said housing.

37. The apparatus of claim 36 wherein said cartridge assembly comprises a plurality of removable plates.

38. The apparatus of claim 36 wherein said cartridge assembly includes a tip well housing comprising a plurality of wells for receiving said fluid carrying members.

39. The apparatus of claim 38 wherein said fluid carrying members include pipette tips.

40. The apparatus of claim 36 wherein the removable assembly further comprises a plurality of vessel retention positions.

41. The apparatus of claim of claim 40 wherein each vessel retention position includes a first opening into which a vessel is inserted and a second opening that is open to the first opening for slidably receiving the vessel from the first opening, wherein the first opening is larger than said second opening.

42. The apparatus of claim 41 wherein said second opening includes securing members for engaging an inserted vessel.

43. The apparatus of claim 41 wherein said assembly further comprises a plurality of cover receiving openings, each for receiving a cover of a respective vessel.

44. The apparatus of claim 38 wherein said head assembly comprises an elongated member for engaging and securely retaining said fluid carrying members.

45. The apparatus of claim 44 wherein said head assembly further comprises an ejector for removing used fluid carrying members from the head assembly.

46. The apparatus of claim 27 wherein said positioning system includes a plurality of travel sensors for limiting the movement of said head assembly in at least one plane.

47. The apparatus of claim 27 wherein said detector system comprises a detector head for determining the presence of an endotoxin within the fluid sample in at least one of the sample holding wells.

48. The apparatus of claim 47 wherein said detector head includes a light source and a detector.

49. The apparatus of claim 48 wherein said light source is an LED.

50. The apparatus of claim 27 further comprising a heating system for providing heat to the removable assembly and a cooling system for maintaining cool temperatures around vessels positioned within said removable assembly.

51. An apparatus for positioning in fluid communication with a fluid line to perform on-line testing of a fluid within the fluid line for the presence of at least one endotoxin, said apparatus comprising:
a housing;
a fluid sampling system for being positioned in fluid communication with the fluid line at a location downstream of a first portion of the fluid line and upstream of a second portion of the fluid line, said fluid sampling system comprising a valve for controlling the fluid flow from the fluid line into the fluid sampling system;
a fluid flow well positioned within said housing for receiving fluid exiting said fluid sampling system;
a removable assembly secured within said housing, said assembly comprising a plurality of wells for receiving used and unused fluid carrying members, a plurality of fluid sample receiving wells, and a plurality of vessel retention positions comprising recesses for securely receiving portions of respective fluid vessels;
a moveable positioning system comprising a head assembly for obtaining the fluid carrying members from said assembly and fluid from said fluid flow well; and
a detection system for determining if an endotoxin is present in a sample within a respective fluid sample receiving well.

52. The apparatus of claim 51 wherein said valve comprises a solenoid valve that operates to control the flow of fluid into said fluid sampling system from the fluid line.

53. The apparatus of claim 52 wherein said fluid sampling system further comprises a fluid holding container downstream of said solenoid valve for receiving the fluid from the fluid line when said solenoid valve is open.

54. The apparatus of claim 53 further comprising a fluid conduit for delivering the fluid from the fluid holding container to said fluid flow well.

55. The apparatus of claim 54 wherein a terminal end of said fluid conduit is spaced from said fluid flow well and said fluid flow well forms a portion of said assembly.

56. The apparatus of claim 51 wherein said fluid flow well comprises an inner trough and an outer trough that surrounds said inner trough and receives fluid overflow from said inner trough.

57. The apparatus of claim 56 further comprising a fluid line extending from said fluid flow well to carry away the overflowed fluid in said outer trough.

58. The apparatus of claim 56 wherein said fluid flow well further comprises a splash guard.

59. The apparatus of claim 51 wherein the removable assembly includes a replaceable cartridge that can be secured within said housing.

60. The apparatus of claim 59 wherein said cartridge comprises at least one removable plate.

61. The apparatus of claim 51 wherein said fluid carrying members include pipette tips.

62. The apparatus of claim of claim 51 wherein each vessel retention position includes a first opening into which a vessel is inserted and a second opening that is open to the first opening for slidably receiving the vessel from the first opening, wherein the first opening is larger than said second opening.

63. The apparatus of claim 62 wherein said second opening includes securing members for engaging an inserted vessel.

64. The apparatus of claim 51 wherein said assembly further comprises a plurality of cover receiving openings, each for receiving a cover of a respective vessel.

65. The apparatus of claim 51 wherein said positioning system comprises a powered arm that carries said head assembly for performing a plurality of functions.

66. The apparatus of claim 65 wherein said head assembly further comprises a member for engaging and retaining the fluid carrying members.

67. The apparatus of claim 66 wherein said head further comprises an ejector for removing fluid carrying members from said head assembly.

68. The apparatus of claim 51 wherein said positioning system further comprises a plurality of travel sensors for limiting the movement of said head assembly in at least one plane.

69. The apparatus of claim 51 wherein said detection system comprises a detector head for determining the presence of an endotoxin within the fluid sample in at least one of said fluid sample receiving wells.

70. The apparatus of claim 69 wherein said detector head includes a light source and a detector.

71. The apparatus of claim 70 wherein said light source is an LED.

72. The apparatus of claim 51 further comprising a heating system for providing heat to the removable assembly and a cooling system for maintaining cool temperatures around vessels within said removable assembly.

73. An on-line endotoxin detection system for being positioned within a fluid line, said system comprising:
means for obtaining fluid from said fluid line;
means for transferring a sample of said obtained fluid to a fluid sample receiving well;
means for combining an agent and said fluid sample together in said well; and
means for detecting the presence of endotoxins within said sample located within said well.

74. The system according to claim 73 wherein said agent comprises a chromogenic substrate and/or a reagent.

75. A method for performing on-line detection of an endotoxin within fluid carried by a fluid line of a fluid system; said method comprising:
positioning an endotoxin testing apparatus within the fluid line of said fluid system, said testing apparatus being in fluid communication with said fluid line;
directing fluid from said fluid line into said testing apparatus;
sampling said directed fluid and delivering the sample to a receiving well;
obtaining an endotoxin identifying agent;
introducing said agent into said receiving well containing said fluid sample; and
detecting the presence of any endotoxin within the sample.

76. The method of claim 75 further comprising positioning a head assembly proximate an instrument, introducing an instrument coupling member into said instrument and securely receiving said instrument on said coupling member.

77. The method of claim 76 wherein said sampling step comprises positioning said instrument within a fluid flow well.

78. The method of claim 77 wherein said sampling step further comprises drawing fluid from within said fluid flow well into said instrument.

79. The method of claim 75 wherein the step of delivering the fluid sample comprises positioning said instrument carrying said fluid sample proximate a fluid receiving well; and depositing at least a portion of the fluid sample into said fluid receiving well.

80. The method of claim 79 wherein said method further comprises positioning said instrument carrying the remainder of said fluid sample proximate a second fluid receiving well; and depositing at least a portion of the remaining fluid sample into said second fluid receiving well.

81. The method of claim 79 further comprising a step of placing and ejecting a used instrument into an instrument retaining well.

82. The method of claim 75 wherein said obtaining step includes removing a cover from a vessel containing said identifying agent; introducing an instrument into the vessel and withdrawing said identifying agent from said vessel.

83. The method of claim 75 wherein said detecting step includes passing a detection assembly and/or a fluorescent reader positioned on a head assembly along the fluid receiving well; passing said head assembly along a fluid receiving well containing a control; and comparing readings from each passing of said head assembly to determine if an endotoxin is present in the fluid sample from said fluid line.

84. The method of claim 75 wherein said detecting step includes measuring fluorescence.

85. The apparatus of claim 2 wherein said chromogenic substrate comprises a fluorogenic substrate.

86. The apparatus of claim 28 wherein said chromogenic substrate comprises a fluorogenic substrate.

87. The system of claim 74 wherein said chromogenic substrate comprises a fluorogenic substrate.
